(19) United States
(12) Patent Application Publication
(10) Pub. No.: US 2004/0228867 A1
(43) Pub. Date: Nov. 18, 2004

(54) Method for screening transcriptional coregulatory proteins of transcription factors, and androgen receptor transcriptional coregulatory proteins as targets for androgen receptor-dependent diseases

(75) Inventors: Michael Garabedian, New York, NY (US); Samir Taneja, Wyckoff, NJ (US); Adam Hittelman, New York, NY (US); Steven Markus, Brooklyn, NY (US)

Correspondence Address: BROWDY AND NEIMARK, P.L.L.C., SUITE 300, 624 NINTH STREET, N.W., WASHINGTON, DC, 20001-5303, US

(73) Assignee: NEW YORK UNIVERSITY, New York, NY

(21) Appl. No.: 10/846,648

(22) Filed: May 17, 2004

Related U.S. Application Data
(62) Division of application No. 09/816,669, filed on Sep. 21, 2001, now abandoned.

(51) Int. Cl. A61K 39/395

(52) U.S. Cl. 424/155.1

(57) ABSTRACT

The present invention also provides a method for screening and isolating transcriptional coregulatory proteins of transcription factors. Using this method, a new class of proteins, androgen receptor transcriptional coregulatory proteins, that interact with the androgen receptor N-terminus to regulate transcriptional activation, which are targets for identifying and isolating inhibitors that disrupt such an interaction, were identified.

### CROSS-REFERENCE TO RELATED APPLICATIONS

The present application is a divisional of application Ser. No. 09/816,669, filed Sep. 21, 2001, which application claims the benefit of priority from U.S. provisional applications No. 60/191,768, filed Mar. 24, 2000, and 60/225,618, filed Aug. 15, 2000, the entire contents of each of the above applications are hereby incorporated by reference.

### BACKGROUND OF THE INVENTION

1. Field of the Invention

The present invention relates to a method for screening transcriptional coregulatory proteins of transcription factors, to androgen receptor transcriptional coregulatory proteins (coactivators and corepressors), and to the use of androgen receptor transcriptional coregulatory proteins as targets for screening compounds that disrupt the interaction between androgen receptor and such coregulatory proteins.

2. Description of the Related Art

The androgen receptor (AR) is a member of the steroid receptor (SR) family of transcriptional regulatory proteins that transduces the signaling information conveyed by androgens (Chang et al., 1995 and Wilson et al., 1991). Upon androgen binding, the androgen receptor is released from the repressive effects of an Hsp90-based regulatory complex, allowing the receptor to either activate or inhibit transcription of target genes in a hormone-dependent manner (Suina et al., 1996; Fang et al., 1996; Fang et al., 1998; Picard et al., 1990; Segnitz et al., 1997; Jenster et al., 1991; and Jenster et al., 1992). In addition to the role the androgen receptor plays in male sex determination, activation of the receptor also mediates normal prostate development and malignant growth by regulating genes involved in cellular proliferation (Brinkmann et al., 1992; Dorkin et al., 1997; Hakimi et al., 1996; Trapman et al., 1996 and Jenster et al., 1999). For example, activation of the androgen receptor is not only responsible for male sexual development, it also plays a critical role in the development and progression of benign prostate hyperplasia, prostate cancer, and hair loss. The androgen receptor controls gene expression through binding with critical transcriptional regulatory proteins (coactivators and corepressors) that, in turn, allow the androgen receptor to “switch on” or “switch off” genes important for malignant prostate cell growth, benign prostate hyperplasia, and androgen-dependent hair loss.

The mechanisms underlying the specificity of androgen receptor regulation of gene expression remain enigmatic. Although the DNA binding domain of androgen receptor is highly conserved among steroid receptors and recognizes the same hormone response element as does the glucocorticoid receptor, recent evidence suggests that the androgen receptor cell- and promoter-specific transcriptional response is generated through interactions with regulatory proteins termed coactivators and corepressors (Scheller et al., 1998 and Cleutjens et al., 1997).

For example, agonist binding to the androgen receptor C-terminal activation function (AF-2) promotes a conformational change and the formation of a surface for protein-protein contacts between AF-2 and additional transcriptional regulatory factors, which in turn modulate the transcriptional activity of target genes (Onate et al., 1995; Smith et al., 1996; Li et al., 1997; Chen et al., 1997; Torchia et al., 1997; Hong et al., 1997; Voegel et al., 1996; Kang et al., 1999 and Yeh et al., 1996). Since the growing number of steroid receptor coactivators and corepressors appear to function widely across the steroid receptor family with conserved regions of AF-2 (Glass et al., 2000), it is unlikely that these factors alone influence receptor specificity. In contrast, the N-terminal transcriptional regulatory regions of steroid receptors, which are diverse throughout the family, may represent an important determinant of steroid receptor specificity, conceivably by recruiting distant coregulators. Indeed, Hittelman et al. recently identified DRIP150 as a glucocorticoid receptor (GR) N-terminal coactivator that does not interact with the N-termini of other steroid receptors, including androgen receptor (Hittelman et al., 1999). However, the mechanisms of transcriptional activation by the androgen receptor N-terminus are not understood, and proteins that specifically associate with it remain largely uncharacterized.

Regions of the androgen receptor N-terminus important for transcriptional activation have been identified by expressing and analyzing receptor deletion derivatives or fusion proteins in mammalian cells and in cell-free systems. At least two distinct activation domains with the androgen receptor N-terminus have been identified, AF-1a (residues 154-167) and AF-1b (residues 295-259), both of which are required for full transcriptional activation mediated by the receptor (Chamberlain et al., 1996). The androgen receptor N-terminus (residues 142-485) has also been shown to activate a minimal promoter construct in a cell-free transcription system and to selectively interact with the transcription factors TFIIF and the TATA-Binding Protein, suggesting a direct contact with the general transcription factors (McEwan et al., 1997). Protein-protein interaction studies have recently suggested contacts between the androgen receptor N-terminus and the TATA-element Modulating Factor (TMF), or ATA160, which increase androgen receptor transcriptional activity when overexpressed in certain cell types (Hsiao et al., 1999). Interestingly, a number of prostate cell lines display elevated androgen receptor-dependent transcriptional activation relative to nonprostatic cell lines, and the androgen receptor N-terminus appears responsible for this enhanced receptor activity (Gordon et al., 1995). These findings suggest the existence of androgen receptor coregulators that modulate transcriptional activation by androgen receptors through the N-terminal activation domain in prostate epithelial cells.

At present, androgen receptor activity can only be altered by removing the hormone, testosterone, by surgical or pharmacological means. Unfortunately, this approach is often short-lived, with androgen-expressing cells “learning” to grow in the absence of testosterone. Once this has occurred, there is no effective treatment for androgen-dependent afflictions.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention provides a method for screening and isolating transcriptional coregulatory proteins of transcription factors, such as the ARTs of the androgen receptor, using a novel “reverse” yeast two hybrid system with a first hybrid protein as bait and a library of second hybrid proteins as prey and screening for the ability to interact with an activation domain of the first hybrid protein as a transcriptional coregulatory protein.

The present invention also provides a new class of androgen receptor transcriptional coregulatory proteins termed ARTs (for Androgen Receptor Trapped) by the present inventors, that interact with the androgen receptor N-terminus, and the DNA encoding such ART proteins.

The present invention further provides for a molecule having the binding portion of an antibody capable of binding to an ART and for an antisense oligonucleotide complementary to the DNA encoding ARTs.

Another aspect of the present invention relates to a method for treating androgen-dependent diseases by administering an effective amount of a molecule having the binding portion of an antibody capable of binding to an ART.

Further aspects of the present invention relate to a method of screening for and identifying inhibitors that disrupt the interaction between androgen receptor and an ART, to an inhibitor obtained by this method, and to a method for inhibiting the interaction between androgen receptor and an ART.

### BRIEF DESCRIPTION OF THE DRAWING

FIGS. 1A and 1B show the results of the modified yeast two-hybrid screen for androgen receptor N-terminus-interacting factors. FIG. 1A shows quantitative analysis of ART interactions with androgen receptor N-terminus and FIG. 1B shows the specificity of androgen receptor-ART interactions.

FIG. 2 shows ART mRNA expression in prostate cancer cells and in human tissues by hybridization to ART-37, ART-27, and ART-5 probes.

FIGS. 3A and 3B shows subcellular localization of ART-27 by indirect immunofluorescence using anti-FLAG primary antibody and rhodamine conjugated secondary antibody (FIG. 3A) and Hoechst fluorescent dye H334211 (FIG. 3B).

FIG. 4 shows immunoblotting with nuclear extracts derived from different indicated cell types using an ART-27-specific polyclonal antibody.

FIG. 5 shows interaction of ART-27 with androgen receptor in vitro as resolved by SDS-PAGE and visualized by autoradiography.

FIGS. 6A and 6B show a quantitative analysis by immunoblot of the domains of androgen receptor and ART-27 mediating interaction.

FIG. 7A and 7B show that ART-27 enhances androgen receptor ligand-dependent and -independent transcriptional activation.

FIG. 8 shows an ART-27 C-terminal deletion derivative (1-127) that fails to interact with androgen receptor is unable to enhance androgen receptor transcription activation.

FIG. 9A shows that the effect of ART-27 on androgen receptor transcription activation depends on the androgen receptor-interacting region and FIG. 9B presents results of a parallel set of transfections analyzed by immunoblotting.

FIG. 10 shows that ART-27 overexpression enhances androgen receptor ligand potency.

FIGS. 11A and 11B show that ART-27 enhances GR (FIG. 11A) and ER (FIG. 11B) alpha-dependent transcriptional activation.

FIG. 12 shows transcriptional activation of ERα or ERβ by ART27 in U2OS cells.

FIGS. 13A and 13B show ART-27 expression in matched normal (N) and tumor tissues (T) for a short exposure (FIG. 13A) or for a long exposure (FIG. 13B).

FIG. 14 shows Western blot analysis of the regulation of ART-27 protein expression in a rat androgen-depletion model with antibodies to PCNA, clustering ART-27 or MAP kinase (MAPK) antibodies.

FIGS. 15A and 15B show expression pattern of endogenous ART-27 in human prostate using immunohistochemical analysis with affinity purified ART-27 antibody (FIG. 15A) and staining (FIG. 15B).

FIG. 16 shows immunoblot analysis of ART-27 protein expression in primary human stromal or epithelial cells.

FIG. 17 shows a schematic representation of a conventional yeast two hybrid system.

FIG. 18 shows a schematic representation of a preferred embodiment of the method using the reverse yeast two hybrid system according to the present invention.

### DETAILED DESCRIPTION OF THE INVENTION

The present inventors have developed an innovative reverse yeast two hybrid system that is generally applicable as a method for screening and isolating transcriptional coregulatroy proteins of transcription factors based on protein-protein interaction as one aspect of the present invention. This method according to the present invention provides a distinct advantage over the conventional yeast two hybrid system because it can be used even when the proteins screened as bait have an activation domain that shows strong transcriptional activity in yeast.

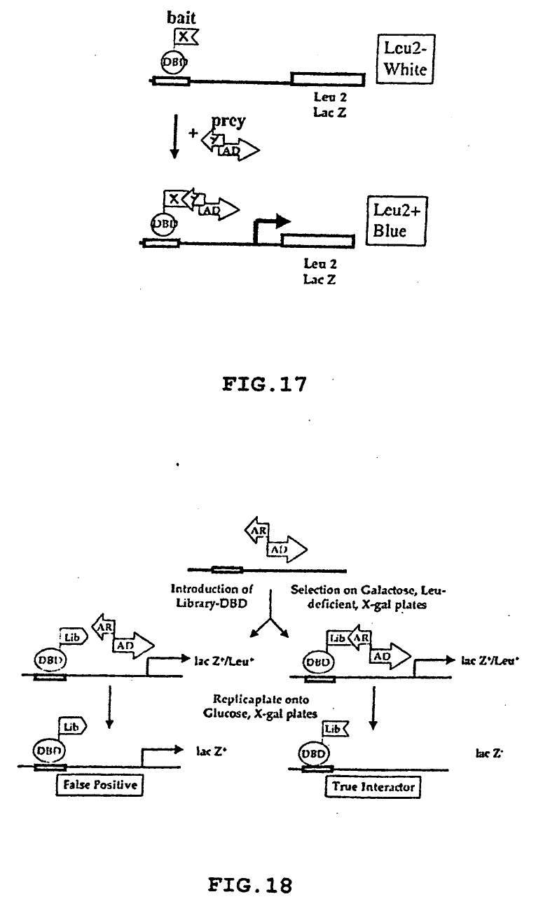

The yeast two hybrid system is a powerful method for identifying protein-protein interactions. A schematic representation of the conventional yeast two hybrid system is presented in FIG. 17. Two hybrid proteins, a “bait” and a “prey”, are generated. The bait hybrid protein is composed of a protein X fused to a DNA binding domain (DBD), whereas the prey hybrid protein is composed of proteins Y fused to a transcriptional activation domain (AD). For this system to work, the bait alone cannot activate transcription of the DNA encoding the reporter (e.g., Leu2, LacZ). If interaction of protein X and Y occurs, a functional transcription activator is generated and results in the transcription of DNA encoding the reporter proteins that confer the Leu⁺ and LacZ⁺ (blue) phenotype. Proteins that intrinsically activate transcription or any protein containing an activation domain which shows strong transcriptional activity in yeast when fused to a DNA binding domain, such as the N-terminal transcriptional activation domain of androgen receptor (AR), are unsuitable as bait in a conventional yeast two hybrid screen and therefore cannot be studied by this conventional method. This is the reason the conventional yeast two hybrid system is precluded from being used to identify transcriptional coregulatory proteins that interact with transcription factors such as AR.

Using the AR as the transcription factor, in particular the N-terminal activation domain of AR which is transcriptionally active in yeast, the present inventors modified the conventional yeast two hybrid system and developed an innovative “reverse” yeast two hybrid system that allows for selection of proteins that interact with transcription factors to isolate transcriptional coregulatory proteins. In this approach, the AR “bait” is created by fusing the N-terminal transcriptional activation domain to a heterologous transcriptional activation domain and the library of “prey” is created by fusing proteins encoded by the cDNA library to a DNA binding domain (rather than to a transcriptional activation domain as is done in a conventional yeast two hybrid system). The DNA binding domain-linked library is then screened for interaction with proteins that are transcription factors.

An embodiment of the reverse yeast two-hybrid system used to identify potential AR interacting proteins according to the method of the present invention is shown in FIG. 18. N-terminal residues 18 through 500 of AR were fused to the B42 activation domain (AD) in a galactose-inducible expression vector as bait. An androgen-stimulated LNCaP (an androgen dependent prostate cancer cell line) cDNA library was fused to the LexA DBD and transformed into yeast cells that expressed the AR₁₈₋₅₀₀-AD fusion and contained the Lex-operator::LEU2 and Lex-operator::LacZ reporter genes. Potential interacting proteins were selected by plating the cDNA library-containing transformants onto galactose plates lacking leucine and containing the chromogenic substrate X-gal. Because some library plasmids may express intrinsic activation domains, rendering them transcriptionally active when fused to DBD (a majority of the colonies contained cDNAs that encode an activation domain, i.e., self-activator false positives, rather than an AR-interacting protein), a second screen was used to eliminate the self-activating false positives. Colonies that grew on galactose in the absence of leucine and expressed LacZ (i.e., blue) were replica-plated onto glucose containing X-gal plates. Since the expression of the AR bait is under the control of the galactose-inducible, glucose-repressible Gal1-10 promoter, potential interactors are blue on galactose (conditions where the AR bait is expressed), but white on glucose-X-gal plates (media where AR is not expressed), whereas false positives are blue on glucose, under which no AR is produced. Clones that activated transcription only in the presence of bait expression (i.e., galactose) were saved, whereas proteins that activated transcription on both glucose and galactose plates were discarded as false positives.

The method for screening and isolating transcriptional coregulatory proteins of transcription factors according to the present invention, of which the above embodiment using androgen receptor as the transcription factor is a preferred embodiment, is generally applicable to transcription factors and can be performed with any suitable transcription factor including, but not limited to, nuclear receptors and steroid receptors. Non-limiting examples of steroid receptors include human estrogen receptor alpha (Green et al., 1986), human estrogen receptor beta (Ogawa et al., 1998), and human progesterone receptor (PR; Kastner et al., 1990); however, it is intended that glucocorticoid receptor, a steroid receptor, be excluded and is therefore not comprehended by the transcription factors for use in the method of the present invention because glucocorticoid receptor is disclosed in Hittelman et al. (1999). Non-limiting examples of nuclear receptors, which are not steroid receptors, include retinoic acid receptor alpha (RAR-alpha; Giguere et al., 1987), thyroid hormone receptor alpha (TR-alpha; Nucleici Acids Res. 15(22):9613, 1987), peroxisome proliferative activated receptor gamma (PPAR-gamma; Elbrecht et al., 1996), and vitamin D3 receptor (VDR; Baker et al; 1988). Also comprehended are those transcription factors which are not steroid or nuclear receptors, such as NF-kappa B (p65; Nolan et al., 1991) and p53 (Harlow et al., 1985).

Even though in the preferred embodiment the activation domain of AR was identified and the N-terminal portion containing the activation domain was used in the hybrid bait protein, knowledge of the location of an activation domain is not needed a priori in order to practice the general screening method for transcriptional coregulatory proteins according to the present invention. Indeed, the entire transcription factor can be used to perform the screen, in order to obtain all the potential interacting proteins, and then deletion mutants of the transcription factor can be used to identify the regions of the transcription factor the interacting proteins interact with. This was the manner in which the laboratory of the present inventors used to obtain transcriptional coregulatory proteins that interact with estrogen receptor alpha and beta.

The method for screening and isolating transcriptional coregulatory proteins of transcription factors using the reverse yeast two hybrid system according to the present invention involves:

fusing a DNA encoding a first transcription factor or a fragment thereof containing a first transcriptional activation domain, which first transcription factor is not a glucocorticoid receptor, to a DNA encoding a second transcriptional activation domain to form a DNA encoding a first hybrid protein as bait on a first yeast expression vector, wherein the expression of the first hybrid protein formed of the first transcription factor or fragment thereof and the second transcriptional activation domain is under the control of a promoter which is inducible in a yeast host cell;

fusing a cDNA from a cell-specific or tissue-specific cDNA library to a DNA encoding a DNA binding domain of a second transcription factor to form a DNA encoding a second hybrid protein as prey on a second yeast expression vector for expression in a yeast host cell;

fusing a DNA encoding a reporter protein to a DNA containing a promoter and a DNA response element, which is the cognate DNA response element for the DNA binding domain of the second transcription factor, to form a reporter gene construct, wherein the expression of the reporter protein is under the control of the promoter and the DNA response element;

transforming auxotrophic yeast host cells with the first yeast expression vector containing the DNA encoding the first hybrid protein as bait, the second yeast expression vector containing the DNA encoding the second hybrid protein as prey, and the reporter gene, together or separately in any order, to generate transformed yeast host cells, wherein the auxotrophic yeast host cells carry a DNA encoding a protein capable of overcoming the auxotrophy of the auxotrophic yeast host cells, the expression of which protein is controlled by a promoter and a DNA response element which is the cognate DNA response element for the DNA binding domain of the second transcription factor;

inducing the expression of the first hybrid protein in the transformed yeast host cells with an inducer;

first screening the transformed yeast host cells for the ability to grow on a culture medium lacking a growth-sustaining component required to complement or overcome the auxotrophy of the auxotrophic yeast host cells and for the ability to express the reporter protein;

screening transformed yeast host cells, which were observed in the first screening to have the ability to grow on a culture medium lacking a growth-sustaining component required to complement or overcome the auxotrophy of the auxotrophic yeast host cells and the ability to express the reporter protein, for the inability to express the reporter protein in the absence of the inducer; and

isolating a transformed yeast host cell identified as being able to express the reporter protein in the presence of inducer but unable to express the receptor protein in the absence of inducer to further isolate a transcriptional coregulatory protein of the first transcription factor and/or its encoding DNA.

As discussed above, the first transcription factor may be any transcription factor including nuclear receptors and steroid receptors with the proviso that it is not glucocorticoid receptor.

A DNA response element, such as the LexA DNA response element used in the preferred embodiment, also commonly known and referred to in the art as upstream activating sequence, enhancer, or operator, and its cognate DNA binding domain are well understood by those of skill in the art of transcriptional regulatory elements/sequences and transcriptional activators. These same skilled artisans would recognize what other suitable DNA response element and cognate DNA binding domain can be used in the present invention.

It will also be appreciated by those of skill in the art that there are many known and well characterized promoters that can suitably be used as the promoter which is inducible by an inducer in yeast. Preferably, the inducible promoter is tightly regulated such that it is only active in the presence of inducer, without being “leaky” in the absence of inducer. However, as would be recognized by those of skill in the art, even “leaky” inducible promoter may be suitable, as long as the level of promoter activity in the absence of promoter is low or negligible, i.e., less than 10-20% of the inducible level. A particularly preferred promoter is the galactose (Gal 1-10) promoter because, not only is it galactose-inducible, it is highly active in the presence of galactose as inducer but inactive (tightly repressed) in the presence of glucose as repressor.

While the preferred reporter protein is β-galactosidase because it is widely used with X-gal as a chromogenic substrate and it is so well-characterized, there are many other well known reporter protein that can also be suitably used in the method of the present invention as would be recognized by those of skill in the art.

Similarly, with auxotrophic (i.e., Leu⁻) yeast host cells and the protein capable of overcoming the auxotrophy (i.e., Leu2), suitable auxotrophic markers and the proteins that are capable of complementing them and overcoming the auxotrophy are well known in the art and would be well recognized by those of skill.

The method for screening and isolating transcriptional coregulatory proteins of transcription factors according to the present invention can use cDNA libraries made from a distinct cell or tissue type to identify cell- or tissue-specific transcriptional coregulatory proteins that interact with transcription factors. For instance, androgen receptor cofactors specific to hair can be identified by using a library generated from dermal papilla cells (hair producing cells that AR regulates).

As another preferred embodiment of the method for screening and isolating transcriptional coregulatory proteins, the present inventors applied the method to estrogen receptor (ER) alpha as the transcription factor. The N-terminal activation domain of ER is transcriptionally active in yeast and cannot be used as a “bait” protein in a conventional yeast two-hybrid screen. To circumvent this problem, the present inventors utilized a modified yeast two-hybrid approach that is capable of isolating proteins that interact with transcriptional activators. Human ER alpha (residues 1-595) subcloned into a galactose-inducible expression vector (pJG 4-5), is expressed as a hybrid protein fused to an acidic B42 transcriptional activation domain (“the bait”). A Hela cell cDNA library cloned into a yeast expression vector (pEG 202) is linked to the LexA DBD (“the prey”) and represents ˜1×10⁷ cDNAs. The auxotrophic yeast strain EGY 188 (trp1 his3 ura3 leu2), with a chromosomally integrated LexA-responsive LEU2 reporter sequence is transformed with 1) the ER bait, the 2) library prey, and 3) a LexA-responsive β-galactosidase (LacZ) reporter sequence. Library proteins that interact with ER (bait-prey interactions) serve to reconstitute transcription and activate LEU2 and LacZ reporter gene expression. Expression of the Lex operator-linked LEU2 reporter allows for auxotrophic EGY 188 cells to grow in the absence of leucine, while β-galactosidase cleaves the chromogenic substrate X-gal, causing the colonies to appear blue. Glucose represses the galactose-inducible promoter, inhibiting production of the ER bait protein. The library was transformed into the strain containing ER and selected for colonies that grew and were blue on galactose, leucine-deficient X-gal plates. Colonies that were blue on galactose X-gal plates, and white on glucose X-gal plates, where no ER is produced, were further analyzed. Using this approach, a number of proteins that interact with ER N-terminal activation domain were identified. Proteins that interact with the ER N-terminal amino acids 1-115 were subjected to an additional screen to identify proteins that specifically associate with ER AF-1.

Through the innovative reverse yeast two hybrid screen, the present inventors have identified a new class of proteins termed Androgen Receptor Trapped proteins, or ARTs, that interact with the N-terminus of the androgen receptor. Using a series of experiments that allows prioritization of the proteins with respect to androgen receptor transcriptional activation, three ART proteins (ART-5, ART-27 and ART-37) have been identified which are important for androgen receptor regulation. All three ART proteins interact strongly with the androgen receptor. In addition, ART-27 and ART-5 increase androgen receptor-dependent transactivation when overexpressed in cultured mammalian cells. Furthermore, ART-27 maps to a region of the X-chromosome amplified in a subset of hormone refractory prostate cancers, suggesting that overexpression of ART-27 may play a role in prostate cancer. Overexpression of ART-27 not only affects ligand efficacy (maximal activation levels at saturating hormone concentrations), but also ligand potency (responding to lower concentration of androgen), indicating that ART-27 plays a key role in determining the sensitivity and activity of androgen receptor to androgen in target cells. Preliminary results in a rat model of androgen-dependent prostate growth demonstrate that the expression of ART-27 protein is dramatically reduced following androgen withdrawal, but is abundant when androgens are available. This suggests that ART27 is regulated by androgens and plays a vital role in AR-mediated transcription and cell growth.

Based on the above discovery, one aspect of the present invention relates to novel proteins, identified and isolated using a reverse yeast two hybrid system, which interact with androgen receptor (particularly near the N-terminus) as androgen receptor transcriptional coregulatory (i.e., coactivator) proteins, and is modified from the conventional yeast two hybrid system used in the art. These novel proteins, termed ARTs, contain the amino acid sequence of SEQ ID NO:4 (ART5), SEQ ID NO:6 (ART37), SEQ ID NO:8 (ART6), or SEQ ID NO:10 (ART2). Also included in this aspect of the present invention are variants of such ARTs which have at least 85% sequence identity, preferably 90% sequence identity and more preferably 95% sequence identity, to any one of the amino acid sequences of SEQ ID NO:4, SEQ ID NO:6, SEQ ID NO:8, or SEQ ID NO:10 and which retain the property of interacting with androgen receptor as androgen receptor transcriptional coregulatory proteins. Common amino acid sequence alignment programs can be used for calculating such high levels (85%, 90%, 95%) of sequence identity because the difference in alignment and calculated % identity between different computer programs would be negligible at such high levels of sequence identity.

Fragments of the ARTs as well as fragments of the ART variants are further encompassed by this aspect of the present invention provided that such fragments retain the property of interacting with androgen receptor as an androgen receptor transcriptional coregulatory protein. It will be appreciated by those of skill in the art that fragments of ARTs are readily obtained by enzymatic or chemical cleavage or by cloning nested deletions generated, for instance, by Bal31 nuclease or other similar acting nucleases.

It should be understood that when the term “antibody” or “antibodies” is used with respect to the antibody embodiment of the present invention, this is intended to include intact antibodies, such as polyclonal antibodies or monoclonal antibodies (mAbs), as well as proteolytic fragments thereof such as the Fab or F(ab′)₂ fragments. Furthermore, the DNA encoding the variable region of the antibody can be inserted into other antibodies to produce chimeric antibodies (see, for example, U.S. Pat. No. 4,816,567) or into T-cell receptors to produce T-cells with the same broad specificity (Eshhar et al., 1990; Gross et al., 1989). Single chain antibodies can also be produced and used. Single chain antibodies can be single chain composite polypeptides having antigen binding capabilities and comprising a pair of amino acid sequences homologous or analogous to the variable regions of an immunoglobulin light and heavy chain (linked V_{H}-V_{L} or single chain Fᵥ). Both V_{H} and V_{L} may copy natural monoclonal antibody sequences or one or both of the chains may comprise a CDR-FR construct of the type described in U.S. Pat. No. 5,091,513 (the entire contents of which are hereby incorporated herein by reference). The separate polypeptides analogous to the variable regions of the light and heavy chains are held together by a polypeptide linker. Methods of production of such single chain antibodies, particularly where the DNA encoding the polypeptide structures of the V_{H} and V_{L} chains are known, may be accomplished in accordance with the methods described, for example, in U.S. Pat. Nos. 4,946,778, 5,091,513 and 5,096,815, the entire contents of each of which are hereby incorporated herein by reference.

A “molecule having the antigen-binding portion of an antibody,” is intended to include not only intact immunoglobulin molecules of any isotype and generated by any animal cell line or microorganism, but also the antigen-binding reactive fraction thereof, including, but not limited to, the Fab fragment, the Fab′ fragment, the F(ab′)₂ fragment, the variable portion of the heavy and/or light chains thereof, and chimeric or single-chain antibodies incorporating such reactive fraction, as well as any other type of molecule or cell in which such antibody reactive fraction has been physically inserted, such as a chimeric T-cell receptor or a T-cell having such a receptor, or molecules developed to deliver therapeutic moieties by means of a portion of the molecule containing such a reactive fraction. Such molecules may be provided by any known technique, including, but not limited to, enzymatic cleavage, peptide synthesis or recombinant techniques.

An antibody is said to be “capable of binding” a molecule if it is capable of specifically reacting with the molecule to thereby bind the molecule to the antibody. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

An “antigen” is a molecule or a portion of a molecule capable of being bound by an antibody which is additionally capable of inducing an animal to produce antibody capable of binding to an epitope of that antigen. An antigen may have one or more than one epitope. The specific reaction referred to above is meant to indicate that the antigen will react, in a highly selective manner, with its corresponding antibody and not with the multitude of other antibodies which may be evoked by other antigens.

The molecule having the antigen binding portion of an antibody according to the present invention can be used for treating an androgen-dependent disease by administering an effective amount of the molecule to a patient in need thereof. Preferably, the administration of an effective amount of the molecule is in the form of a composition which includes a pharmaceutically acceptable excipient, diluent, carrier or auxiliary agent. Non-limiting examples of androgen-dependent diseases or diseases in which specific ARTs may have clinical relevance include prostate cancer, benign prostatic hyperplasia (BPH), androgen-dependent hair loss, age-related alopecia, polycystic ovary disease, AR related intersex disorders such as hypogonadism, testicular feminization, or 5-alpha reductase deficiencies, and age-related hypogonadal effects such as loss of muscle mass or fatigue. In the most common clinical disorders of increased androgen stimulation such as prostate cancer, BPH, and hair loss, the therapeutic strategy would require disruption of ART to AR interaction. This could be achieved with antibodies or could be potentially achieved through small molecules that disrupt of ART-AR interaction or through gene therapy approaches to affect AFT expression, such as creation of dominant negative ARTs, or antisense RNA inhibition of ART expression. In cases of decreased androgen stimulation such as age-related hypogonadal states, ARTs could be overexpressed to increase AR activity while avoiding the potentially carcinogenic effects of exogenous androgens on the prostate.

The present invention also provides for an isolated nucleic acid molecule, i.e., DNA molecule, which includes a nucleotide sequence that encodes for an ART containing any one amino acid sequence of SEQ ID NO:4, SEQ ID NO:6, SEQ ID NO:8, or SEQ ID NO:10. The nucleotide sequence preferably contains any one of SEQ ID NO:3 (ART5), SEQ ID NO:5 (ART37), SEQ ID NO:7 (ART6), or SEQ ID NO:9 (ART2). Also encompassed by the present invention are a self-replicable vector carrying the DNA molecule encoding an ART, a host cell, which can be either prokaryotic or eukaryotic, transformed with the ART encoding DNA molecule, and a process for producing an ART. The process for producing an androgen receptor transcriptional coregulatory protein, which is also known as ART, involves cultivating the host cell transformed with the DNA encoding ART to produce the ART protein and then recovering the produced ART protein.

Another aspect of the present invention relates to an antisense oligonucleotide complementary to a messenger RNA transcribed from the DNA molecule encoding an ART. This antisense oligonucleotide inhibits the production of an ART protein which interacts with the androgen receptor and is preferably a DNA oligonucleotide. The length of the antisense oligonucleotide is preferably between 9 and 150, more preferably between 12 and 60, and most preferably between 15 and 50 nucleotides. Suitable antisense oligonucleotides that inhibit the production of the ART protein of the present invention from its encoding mRNA can be readily determined with only routine experimentation through the use of a series of overlapping oligonucleotides similar to “gene walking” techniques that are well-known in the art. Such “walking” techniques as well-known in the art of antisense development can be done with synthetic oligonucleotides to walk along the entire length of the sequence complementary to the mRNA in segments on the order of 9 to 150 nucleotides in length. This “gene walking” technique will identify the oligonucleotides that are complementary to accessible regions on the target mRNA and exert inhibitory antisense activity.

Alternatively, an oligonucleotide based on the coding sequence of an ART protein which interacts with the androgen receptor N-terminus can be designed using Oligo 4.0 (National Biosciences, Inc.). Antisense molecules may also be designed to inhibit translation of an mRNA into a polypeptide by preparing an antisense which will bind in the region spanning approximately −10 to +10 nucleotides at the 5′ end of the coding sequence.

The mechanism of action of antisense RNA and the current state of the art on use of antisense tools is reviewed in Kumar et al., (1998). The use of antisense oligonucleotides in inhibition of BMP receptor synthesis has been described by Yeh et al., (1998). The use of antisense oligonucleotides for inhibiting the synthesis of the voltage-dependent potassium channel gene Kv1.4 has been described by Meiri et al., (1998). The use of antisense oligonucleotides for inhibition of the synthesis of Bc1-x has been described by Kondo et al., (1998).

The therapeutic use of antisense drugs is discussed by Stix in Sci. Amer. 279, p. 46, 50, 1998, Flanagan, Cancer Metastasis Rev. 17, p. 169-76, 1998, Guinot and Temsamani, Pathol. Biol. (Paris) 46, p. 347-54, 1998, and references therein.

Modifications of oligonucleotides that enhance desired properties are generally used when designing antisense oligonucleotides. For instance, phosphorothioate bonds are used instead of the phosphoester bonds that naturally occur in DNA, mainly because such phosphorothioate oligonucleotides are less prone to degradation by cellular enzymes. Peng et al. teach that undesired in vivo side effects of phosphorothioate oligonucleotides may be reduced when using a mixed phosphodiester-phosphorothioate backbone. Preferably, 2′-methoxyribonucleotide modifications in 60% of the oligonucleotide is used. Such modified oligonucleotides are capable of eliciting an antisense effect comparable to the effect observed with phosphorothioate oligonucleotides. Peng et al. teach further that oligonucleotide analogs incapable of supporting ribonuclease H activity are inactive.

Therefore, the preferred antisense oligonucleotide of the invention has a mixed phosphodiester-phosphorothioate backbone. Preferably, 2′-methoxyribonucleotide modifications in about 30% to 80%, more preferably about 60%, of the oligonucleotide are used.

In order to be effective as a therapeutic, the antisense oligonucleotides of the present invention must travel across cell membranes. In general, antisense oligonucleotides have the ability to cross cell membranes, apparently by uptake via specific receptors. As the antisense oligonucleotides are single-stranded molecules, they are to a degree hydrophobic, which enhances passive diffusion through membranes. Modifications may be introduced to an antisense oligonucleotide to improve its ability to cross membranes. For instance, the oligonucleotide molecule may be linked to a group which includes partially unsaturated aliphatic hydrocarbon chain and one or more polar or charged groups such as carboxylic acid groups, ester groups, and alcohol groups. Alternatively, oligonucleotides may be linked to peptide structures, which are preferably membranotropic peptides. Such modified oligonucleotides penetrate membranes more easily, which is critical for their function and may therefore significantly enhance their activity. Palmityl-linked oligonucleotides have been described by Gerster et al., (1998). Geraniol-linked oligonucleotides have been described by Shoji et al., (1998). Oligonucleotides linked to peptides, e.g., membranotropic peptides, and their preparation have been described by Soukchareun et al., (1998). Modifications of antisense molecules or other drugs that target the molecule to certain cells and enhance uptake of the oligonucleotide by said cells are described by Wang, (1998).

Drug development efforts entail an iterative process of isolating small molecules with a desired biological or biochemical property, defining the mechanism of action and refining the structure to achieve more specific or potent effects. As information accumulates about the role coactivators and corepressors play in regulating transcriptional activity of androgen receptor (AR), it is of interest to develop small molecules that modulate protein-protein interactions as potential therapeutic agents. Thus, a further important aspect of the present invention relates to a method of screening for and identifying inhibitors that disrupt the interaction between androgen receptor and an androgen receptor transcriptional coregulatory protein.

To identify cell-permeating small molecules that target AR_{AF-1}-ART interaction, a high throughput β-galactosidase assay based on the modified yeast two-hybrid system can be utilized as one embodiment of the present method. By adapting the growth and assay of yeast to a 96-well microtiter format, quantitative data from a large number of samples can be generated with minimal effort and reagent expenditure. For example, a library containing 15,000 compounds that consists of a set of structurally diverse small molecules (300-500 daltons) that vary in functional groups and charge can be initially screened. This library is available commercially from Chembrige Corporation (Diverse E) and represents a unique set of small molecules, rationally preselected to form a “universal” library that yields the maximum diversity with the minimum number of compounds. This library is geared for primary screening against a wide range of biological targets, including those where no structural information is available. Recently, a compound from this library has been used successfully to isolate a novel inhibitor of mitotic spindle formation.

A 100 μl volume of a log phase culture of yeast containing AR_{AF-1} and ART will be dispensed into round bottom 96-well microtiter plate preloaded with 5 μl of the compound (5 μg/ml in DMSO) to be tested, treated for 8 hours, and the β-galactosidase activity will be measured using a temperature controlled microtiter plate reader. Those compounds that inhibit AR-ART interaction will have lower β-galactosidase activity than mock treated control cells and will be analyzed further. 1000 compounds a week can be easily assayed using this format. An inherent problem with this type of screen is the ability of yeast cells to take up the compound. To circumvent this potential problem, yeast mutants with increased permeability or higher general uptake, such as the erg6 strain, can be used.

A two-hybrid system adapted for use in mammalian cells, such as the CHECKMATE mammalian two-hybrid system (Promega, Madison, WI) described in Promega Technical Manual No. 049, revised June 2000, which is available at www.promega.com and is incorporated herein entirely by reference can also be employed to identify small molecules that disrupt AR-ART interaction. In this system, for instance, ART-27 is cloned into a vector that encodes the Gal4 DNA binding domain and AR AF-1 is placed upstream of the herpes simplex virus VP16 activation domain to generate fusion proteins. The pGAL4-ART97 and pVP16 ARAF-1 are transfected into HeLa cells (or CHO, 293, PC3 mammalian cells) along with a pG5 luciferase (reporter gene containing five Gal4 binding sites upstream of a minimal TATA box, which in turn is upstream of the firefly luciferase gene). Two to three days after transfection, the cells are lysed and the amount of luciferase is quantitated. Interaction between ART-27 and AR fusion proteins results in an increase in luciferase expression over the negative control. The growth and luciferase assay of mammalian cells can be adapted to a 96-well microtiter format and a library that consists of a set of structurally diverse small molecules (300-500 daltons) that vary in functional groups and charge can be initially screened. A 50,000/well of mammalian cells will be transfected with pGAL4-ART27 and pVP16 AR_{AF-1} along with pG5 luciferase reporter construct, and 2-24 hours later, will be treated with 5 μl of the potential inhibitor compound (5 μg/ml in DMSO) to be tested for 8-48 hours and the luciferase activity will be measured. Those compounds that inhibit AF-ART interaction will have lower luciferase activity than mock treated control cells and will be analyzed further.

Potential false positives are also expected from such in vivo screening methods and include generalized toxicity, inhibitors of LacZ or luciferase reporter gene expression or enzymatic activity, general transcription inhibitors, and DNA binding inhibitors. Such nonspecific compounds could be eliminated in a secondary screen involving unrelated proteins interacting in the context of the two-hybrid system. Alternatively, a variation of the two-hybrid assay in which disruption of a protein-protein interaction has been developed and is designated the spilt-hybrid system. This approach permits the identification of molecules that abrogate or “split” the association of two interacting protein. In the present invention, activation of a reporter gene would result from the dissociation of AR_{AF-1}-ART interaction and should eliminate potential false positives due to toxicity in the conventional assay. The split-hybrid system may also provide a greater degree of sensitivity, allowing the detection of compounds that only moderately affect AR-ART interactions. The split-hybrid system will be employed if a large number of false positives are identified using the modified yeast two-hybrid system. As an additional test for specificity, whether or not molecules that dissociate AR-ART interaction in yeast also disrupt protein-protein interaction in vitro, using a GST pull-down assay described previously will be examined. It is anticipated that prototype compounds that disrupt AR-ART interaction in the yeast two-hybrid assay should also dissociate the interaction in a GST pull-down experiment.

Alternatively, dissociating peptides using the modified yeast two hybrid system can also be identified. Currently, peptides are typically not useful as therapeutics due to their poor stability and problems inherent in their delivery. However, peptides can be used as lead molecules for chemical design of small organic molecules and also can be used in functional studies.

The effect of such prototype molecules on sequence-specific transcriptional activation by AR will be examined. PC3 cells will be transfected with CMV-hAR, an ARE-linked luciferase reporter gene and treated with the AR-ART inhibitor for 8 hours or with vehicle control, and reporter gene activity will be measured in the presence and absence of the synthetic androgen R1881. It is anticipated that molecules that disrupt AR-coactivator interaction reduce AR transactivation. Toxicity of the compound toward mammalian cells will also be monitored via morphological observation, cellular proliferation assays and through the use of vital stain. If toxicity is apparent, then shorter treatment regimes will be employed. Whether or not the prototype compound can inhibit the AR-dependent growth of LNCaP cells in culture will also be examined.

While other suitable methods of screening for and identifying inhibitors of AR-ART interaction as coactivator assays are intended to be encompassed, the present invention preferably utilizes some form of a two-hybrid system, be it a yeast based system, such as the system described in Hittelman et al. (1999), or a mammalian based system, such as the CHECKMATE mammalian two-hybrid system of Promega Corp., Madison, Wis. The basis of two-hybrid systems as a commonly used method for detecting protein to protein interactions in vivo, is the modular domains found in some transcription factors, i.e., a DNA-binding domain, which binds to a specific DNA sequence, and a transcriptional activation domain, which interacts with the basal transcriptional machinery. A transcriptional activation domain in association with a DNA-binding domain may promote the assembly of RNA polymerase II complexes at the TATA box and increase transcription. For example, the DNA-binding domain and the transcriptional activation domain, which may be produced by separate plasmids, are closely associated when one protein fused to a DNA-binding domain interacts with a second protein fused to a transcriptional activation domain such that interaction of the first protein with the second protein, i.e., AR with ART, results in transcription of a reporter sequence or a selectable marker sequence.

In the method of screening for and identifying inhibitors that disrupt AR-ART interaction, androgen receptor and ART protein, such as an ART protein containing an amino acid sequence of SEQ ID NO:2, SEQ ID NO:4, SEQ ID NO:6, SEQ ID NO:8, SEQ ID NO:10, SEQ ID NO:12, or SEQ ID NO:14, are incubated with or without a potential inhibitor. The potential inhibitor is identified as an inhibitor when the level of activity of a receptor gene product or a selectable marker gene product in the presence of the potential inhibitor is substantially less than the level of activity of the same reporter or marker gene product in the absence of the potential inhibitor. This inhibitor, once identified, can be isolated. Both the human and the rat androgen receptor can be suitably used in this method because the rat and human androgen receptors are very similar. The rat androgen receptor was observed to function indistinguishably in human and rodent cells, suggesting that the factors utilized by the receptor are conserved between species.

The present invention further provides for an inhibitor isolated according to the method of the present invention as well as a method of using this inhibitor to inhibit the interaction between androgen receptor and an androgen receptor transcriptional coregulatory protein.

Having now generally described the invention, the same will be more readily understood through reference to the following example which is provided by way of illustration and is not intended to be limiting of the present invention.

### EXAMPLE

### Experimental Procedures

Construction of Plasmids

Yeast expression vectors for the LexA-AR fusion protein, LexA-AR₁₈₋₅₀₀, were created by digesting the rat AR N-terminus with EcoRI and XhoI and subcloned into pEG202 vector digested with EcoRI and XhoI. The subregions of the rat AR N-terminus (LexA-AR₁₈₋₁₅₆, LexA-AR₁₅₃₋₃₃₆ and LexA-AR₃₃₆₋₅₀₀) were subcloned from LexA-AR₁₈₋₅₀₀ as follows: for LexA-AR₁₈₋₁₅₆, pEG202:AR₁₈₋₅₀₀ was digested with EcoRI and PvuII and the insert was ligated into pEG202 digested with NotI, the 5′ overhang filled in with DNA polymerase Klenow fragment to create a blunt end, and EcoRI; for LexA-AR₁₅₃₋₃₃₆, pEG202:AR₁₈₋₅₀₀ was digested with BstYI and AflII, the ends were filled in with Klenow, and the insert was ligated into pEG202 digested with BamHI and XhoI with ends filled in; for LexA-AR₃₃₆₋₅₀₀, pEG202:AR₁₈₋₅₀₀ was digested with BstYI and XhoI and the insert was ligated into pEG202 digested with BamHI and XhoI. To express these fusion proteins in a mammalian system, the LexA DNA-binding domain AR N-terminal fusions from PEG202 were subcloned by digestion with HindIII and XhoI, and the insert was ligated into pcDNA3 digested with HindIII and XhoI. Yeast two-hybrid ‘bait’ proteins, B42-AR₁₈₋₁₅₆, B42-AR₁₅₃₋₃₃₆, B42-AR₃₃₆₋₅₀₀ and B42-AR₁₈₋₅₀₀ were constructed by subcloning respective EcoRI-XhoI fragments from pEG202 into the corresponding sites in pJG4-5. The LexA-LNCaP cell cDNA library was purchased from Origene Technologies, Inc (Rockville, Md.). The rat AR ligand binding domain (AR₅₇₉₋₉₀₁) was amplified by PCR using the following primers: forward primer with a BglII site, 5′-AGATCTTAAGCAGAAATGATTGCACCATTG-3′ (SEQ ID NO:15); reverse primer with a XhoI site, 5′-GTAGATAAAGGTGTGTGTCACTGAGCTC-3′ (SEQ ID NO:16). The PCR product was ligated into pGEM:T-easy (Promega Corporation, Madison, Wis.) and digested with BglII and XhoI, and the insert was ligated into pEG202 digested with BamHI and XhoI. pEG202:AR₅₇₉₋₉₀₁ was then digested with EcoRI and XhoI and the insert was ligated into pJG4-5 digested with EcoRI and XhoI.

The LexA-ART-27 C-terminal truncations 1-45, 1-67, and 1-127 were constructed by digesting pEG202:ART-27 with PvuII, BspMI and StyI, respectively, filling in their 5′ overhangs with Klenow, digesting with MluI (upstream pEG202 site) and ligating the inserts into pEG202 digested with NotI, the 5′ overhang filled in, and subsequently, MluI. The LexA-ART-27 N-terminal truncations 46-157, 68-157 and 127-157 were constructed by digesting pEG202:ART-27 as follows: for LexA-ART-27₄₆₋₁₅₇, pEG202:ART-27 was digested with PvuII and XhoI and the insert was ligated into pEG202 digested with BamHI, the 5′ overhang filled in with Klenow, and XhoI; for LexA-ART-27₆₈₋₁₅₇, pEG202:ART-27 was digested with BspMI, the 5′ overhang filled in with Klenow, and XhoI, and the insert was ligated into pEG202 digested with BafnHI, the 5′ overhang filled in with Klenow, and XhoI; for LexA-ART-27₁₂₇₋₁₅₇, pEG202:ART-27 was digested with StyI, the 5′ overhang filled in with Klenow, and XbaI, and the insert was ligated into pEG202 digested with EcoRI, the 5′ overhang filled in, and XbaI. For LexA-ART-27_{1-45/127-157}, PCR primers were designed as follows: ART-27₁₋₄₅ forward pEG202 primer, 5′-TTGGGGTTATTCGCAACGG-3′ (SEQ ID NO:17), reverse primer with BamHI site,

| | |
|---|---|
| 5′-GAACTGGATCCCTGCTCATATACCTTG | (SEQ ID NO: 18) |
| TCTCGATG- 3′; | |

ART-27₁₂₇₋₁₅₇ forward primer with BamHI site 5′-GAACTGGATCCACCAAGGACTCCATG-3′ (SEQ ID NO:19); reverse pEG202 primer, 5′-CGGAATTAGCTTGGCTGC-3′ (SEQ ID NO:20). The two separate fragments were amplified via PCR and the resulting products were digested as follows: ART-27₁₋₄₅ with EcoRI and BamHI, ART-27₁₂₇₋₁₅₇ with BamHI and XhoI, and the two inserts were ligated together into pEG202 digested with EcoRI and XhoI.

The two ART-27 derivatives used in the mammalian cell culture experiments were constructed as follows: using EcoRI-XhoI, ART-27 was subcloned from pEG202:ART-27 into a pcDNA3 vector that has an N-terminal HA epitope (pCDNA3-HA) in the same reading frame as the LexA moiety in pEG202 with respect to the EcoRI site; ART-27₁₋₁₂₇ was subcloned from pEG202:ART-27₁₋₁₂₇ into pcDNA3-HA, pJG4-5:Sp1₈₃₋₂₆₂, pJG4-5:Sp1₂₆₃₋₅₄₂, pJG4-5:TAF130₂₇₀₋₇₀₀, and pJG4-5:CREB₃₋₂₉₆ were provided by N. Tanese (New York University School of Medicine, New York). pJG4-5:SRC-1₃₇₄₋₈₀₀ was provided by H. Samuels (New York University School of Medicine, New York). pJG4-5:GR₁₀₇₋₂₃₇ was previously described (Hittelman et al., 1999). The pJK103 reporter plasmid, which contains a single LexA operator linked to β-galactosidase, was used in all activity assays of the LexA fusion proteins and in the modified two-hybrid assay. The pΔ4X-LALO-luciferase reporter plasmid, which contains four LexA operators upstream of a minimal Drosophila alcohol dehydrogenase promoter linked to luciferase, was used in mammalian activity assays to monitor the intrinsic transcriptional activity of the LexA fusion proteins. The pcDNA3:hAR expression plasmid was used to produce full length human AR, pMMTV:luciferase reporter was used to assay AR transcriptional activity, while pCMV:LacZ constitutively expressed β-galactosidase, a marker for efficiency of transfection.

Modified Yeast Two-hybrid Approach

The modified yeast two hybrid assay is described in Hittelman et al., 1999. EGY188 was transformed by the lithium acetate method with (i) pJG4-5:AR₁₈₋₅₀₀, (ii) pEG202:LNCaP cell cDNA library and (iii) pJK103, a β-galactosidase reporter gene with a single LexA operator. Potential interacting proteins were selected by plating the CDNA library expressing transformants onto galactose plates lacking leucine and containing X-gal.

Quantitative Liquid β-galactosidase Assay

Yeast were grown in selective liquid media containing 2% glucose for approximately 12 hours, pelleted, washed once with sterile H₂O, normalized according to cell number and resuspended to an optical density (OD₆₀₀) of 0.15 in 2% galactose/1% raffinose. β-galactosidase assays were performed 12 hours later as described previously (Garabedian et al., 1992).

Northern Blotting

Cells were cultured in 100 mm dishes for indicated periods of time with appropriate treatments, the media aspirated and cells lysed directly on the dishes by adding 3 ml/dish of RNA STAT-60 reagent (Tel-Test, Inc., Friendswood, Tex.). Total RNA was isolated from cell homogenates as per the manufacturer's instructions, denatured at 65° C for 15 min, chilled on ice and separated on a 1.2% agarose -6% formaldehyde denaturing gel (10 μg RNA/lane). Equivalent loading was verified by ethidium bromide staining of ribosomal RNA. RNA was transferred to “Duralon” (Stratagene, San Diego, Calif.), UV-crosslinked to the membrane and hybridized to a cDNA probe using QuikHyb hybridization mix (Stratagene, San Diego, Calif.) as described by the manufacturer. cDNA fragments encoding ART-5, -27 and -37 were labeled with [α-³²P] dCTP using RediPrime random priming labeling kit (Amersham Pharmacia Biotech, Piscataway, N.J.) using the manufacturer's instructions. Blots were washed and exposed to Kodak BioMax film at −80° C for autoradiography. Hybridization of ARTs to multiple tissue northern blot membrane (Clontech, Palo Alto, Calif.) was performed as per the manufacturer's instructions.

In vitro Co-immunoprecipitation

Full length AR and HA-ART-27 were translated in vitro using TNT Quick Coupled Transcription/Translation System (Promega, Madison, Wis.) in the presence of [³⁵S]-methionine. The radiolabeled proteins were incubated as indicated in binding buffer (20 mM Tris pH7.9, 170 mM KCl, 20% glycerol, 0.2 mM EDTA, 0.05% Nonidet P-40 (NP-40), 0.1 mM phenylmethylsulfonyl fluoride (PMSF), 1 mM dithiothreitol (DTT) and 4 mg/ml bovine serum albumin (BSA)) for 1 hour at 42C. 1 μg of α-HA (12CA5) antibody (Boehringer Mannheim, Indianapolis, Ind.) was incubated with the radiolabeled proteins for 1 hour at 4° C. 30 μl of Protein A Sepharose Fast Flow beads (Amersham Pharmacia Biotech) were incubated with the respective reaction mixes for an additional hour at 4° C. The beads were washed three times in wash buffer (20 mM Tris pH 7.9, 170 mM KCl, 20% glycerol, 0.2 mM EDTA, 0.05% NP-40), resuspended in 2X SDS sample buffer and boiled for 3 minutes; the associated proteins were resolved by SDS-PAGE and visualized by autoradiography.

Mammalian Cell Culture and Transient Transfection Assays

A human cervical carcinoma cell line (HeLa), a human prostate cancer cell line (PC-3), and an SV40 T-antigen expressing monkey kidney cells (COS-1) cells were obtained from the ATCC and maintained in Dulbecco's modified Eagle's Medium (DMEM; Life Technologies, Grand Island, N.Y.) supplemented with 10% fetal bovine serum (FBS; HyClone Laboratories, Inc., Logan, Utah), 50 U/ml each of penicillin and streptomycin (Life Technologies) and 2 mM L-glutamine (Life Technologies). The androgen-dependent prostate cancer cell line (LNCaP) was maintained in RPMI-1640 (Life Technologies) supplemented with 10% FBS, 50 units/ml each of Penicillin and Streptomycin and 2 mM L-Glutamine. For transfections, HeLa cells were seeded in 35 mm dishes at a density of 1.3×10⁵, washed once with serum-free medium and transfected with 0.2 μg pcDNA3:hAR, 0.1 μg pMMTV-Luc, 0.05 μg pCMV-LacZ, and the indicated concentrations of pcDNA3:HA-ART-27, or derivative thereof, using 5 μl of lipofectamine reagent (Life Technologies) in a total volume of 1 ml of serum-free, phenol red-free DMEM per 35 mm dish according to the manufacturer's instructions. Approximately four hours post-transfection, the transfection mix was removed, the cells were refed with 2 ml of DMEM-10% FBS, allowed to recover for 3-5 hours, and were fed again with fresh DMEM-10% FBS supplemented with 100 nM R1881 or an identical volume of 100% ethanol and incubated for 12 hours. Transfected cells were washed once in phosphate-buffered saline and harvested in 1X reporter lysis buffer (Promega) as per the manufacturer's instructions. PC-3 cells were seeded in 35 mm dishes at a density of 1.1×10⁵ and transfected as above. To assay LexA-AR N-terminus derivatives in HeLa cells, 0.5 μg pcDNA3-LexA:AR N-terminus derivative, 1.0 μg pCDNA3-HA:ART-27, or empty vector, 1.0 μg pΔ4X-LALO-Luc reporter, and 0.25 μg pCMV-LacZ were transfected using 6 μl of lipofedtamine. Luciferase activity was quantitated in a reaction mixture containing 25 mM glycylglycine, pH 7.8, 15 mM MgSO₄, 1 mM ATP, 0.1 mg/ml BSA, 1 mM DTT using a Lumen LB 9507 luminometer (EG&G Berthold) and 1 mM D-luciferin (Pharmingen) as substrate.

Immunoblotting

Yeast protein extracts were prepared from 2 ml cultures and lysed using glass beads as previously described (Knoblauch et al., 1999). Lysates from mammalian cells were prepared as described in Hittleman et al., (1999). Extracts were normalized according to the Bradford protein assay (Bio-Rad) and separated on SDS—4-20%polyacrylamide gels (Novex) and transferred to Immobilon paper (Millipore). Membranes were probed with a polyclonal antibody against LexA (a gift from E. Golemis) or a monoclonal antibody to HA (12CA5; Boehringer Mannheim). The blots were developed using horseradish peroxidase-coupled donkey anti-rabbit or sheep anti-mouse antibodies and enhanced chemiluminescence (ECL) (Amersham-Pharmacia).

{tc \11″}Subcellular Localization of AH-ART27

Hela cells were seeded onto poly-D-lysine coated cover slips, transfected with pcDNA3-HA-ART-27, and 24 hours later, the cells were washed 5 times with PBS and fixed in 4% paraformaldehyde in PBS for 20 min at room temperature. Cells were then permeabilized by incubating with 0.2% Triton X-100 (Bio-Rad Laboratories, Hercules, Calif.) in PBS and then incubated with 100 μl of the HA-antibody (12CA5) diluted to a concentration of 2 μg/ml in blocking solution (5% BSA/TBS) for 2 hours at room temperature. Cells were washed five times in 1 ml of Triton X-100 in PBS, followed by incubation with goat anti-mouse rhodamine-conjugated secondary antibody (Vector Labs), diluted in blocking solution, for four hours at room temperature. Secondary antibody was removed by washing the cells five times in PBS. To visualize nuclei, cells were then incubated in 1 μg/ml of Hoechst dye H334211 for 10 minutes, followed by one wash with PBS. Cover slips were mounted onto Citifluor (Ted Pella, Redding, Calif.), and the fluorescein and Hoechst signals were visualized and photographed using a Zeiss Axioplan 2 microscope.

Immuno-histochemistry Protocol for Staining Prostate Tissue with Polyclonal Affinity Purified Rabbit ART-27-antibody

The protocol for immunohistochemical staining of prostate tissue with polyclonal affinity purified rabbit ART-27 antibody is as follows:

1. Use 5-7 micrometer thick tissue sections on charged slides.

2. Deparaffinization sequence: Xylene 3min×4 washes, 100% EtOH 3min×2 washes, 95% EtOH 3min×2 washes, rinse in distilled H₂O.

3. Antigen retrieval with Target retrieval solution from DAKO sold as 10× premade solution that needs to be diluted to 1x, 500cc is generally sufficient. Samples placed in microwave for 15 minutes.

4. Remove samples from microwave and cool down to room temperature; use cold room to facilitate this step.

5. 3% hydrogen peroxide for 10-15 minutes

6. Rinse in dH₂O

7. Apply PAP pen around the tissue on the slide and place in 1x PBS (Shandon Cadenza buffer preferred delivered as 30 ml volumes that need to be diluted with 970ml of dH2O.) for 3-5 minutes.

8. Block tissue with 20% normal goat serum for 30 minutes

9. Apply primary ART-27 antibody 1:100 dilution for 35-40 minutes at room temperature

10. 1x PBS 5min×3 washes

11. Apply secondary antibody (Vector anti-rabbit affinity purified) 1:200 dilution for 30 minutes

12. 1x PBS 5min×3 washes

13. Streptavidin orange (Biomeda) 1-2 drops per slide for 30 minutes.

14. 1x PBS 5min×3 washes

15. DAB staining (follow instructions in the kit) for 60-90 seconds in the dark.

16. 1x PBS quick 3 washes

17. Rinse in dH2O

18. Hemotaxylin 1 min followed with running water

19. Acid alcohol 2-3 dips followed with running water

20. Ammonia water 2-3 dips followed with running water

21. Drying sequence: 95% EtOH 3min×2 washes, 100% EtOH 3min×2 washes, Xylene 3min×4 washes.

22. Cover tissue with “Premium Cover Glass” cover slips from Fisher 24×50mm.

### Results

To identify proteins that interact with the androgen receptor N-terminus, a modified yeast two-hybrid system that allows one to identify factors expressed in the prostate which associate with transcriptional activators was used. An androgen-stimulated LNCaP prostate cancer cell cDNA library fused to the LexA DNA binding domain was screened for proteins that interact with the androgen receptor N-terminal transcriptional activation domain encompassing receptor residues 18 through 500 (using the rat androgen receptor number scheme). This library was used to search for androgen receptor interacting proteins for several reasons. First, this library is prostate-specific, being derived from a well-characterized androgen receptor-expressing androgen-dependent prostate cancer cell line. Second, androgen receptor in LNCaP cells activates transcription of a bona fide androgen receptor-responsive gene (e.g., PSA), which implies that the androgen receptor cofactors required for its regulation are present. Third, choosing androgen-stimulated LNCaP cells as the source of mRNA from which the library was produced also allows for the enrichment and detection of androgen-inducible androgen receptor-associated factors. In principle, androgen-regulated androgen receptor-interacting cofactors may represent a means through which androgen receptor-dependent transcriptional activity is modulated. Finally, since LNCaP cells are androgen-dependent for growth, the use of this library increases the likelihood of identifying cofactors that regulate the androgen receptor mitogenic response.

Out of approximately one million library transformants, eight clones were isolated that interact with the androgen receptor N-terminus. There protein factors were termed ARTs, for Androgen Receptor Trapped, by the present inventors. The eight ART clones were sequenced and were subjected to a database search using the BLAST program. A quantitative liquid beta-galactosidase assay was used to measure the relative strength of interaction between the androgen receptor N-terminus and the ARTs using the yeast two-hybrid system. The levels of expression of the ARTs in yeast were similar, as determined by immunoblotting using an antibody to the LexA DNA-binding domain that is common to all of the ARTs.

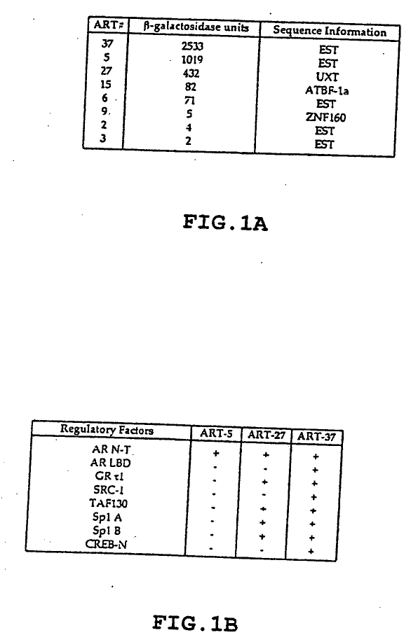

FIG. 1A shows the results of the search of the NCBI and Swissprot databases using the BLAST search program for homologies to known proteins and quantitative analysis of the relative strength of ART interactions with androgen receptor N-terminus. ARTs expressed as fusion proteins with the LexA DNA binding domain were analyzed for their ability to interact with AR18-500. The relative strength of interaction was determined by a quantitative liquid beta-galactosidase assay after a twelve hour incubation in galactose-containing media at 30° C. The LexA vector alone gives 1 unit of activity.

The strongest androgen receptor N-terminal interacting proteins, in decreasing order of affinity, are ART-37, ART-5, and ART-27. Art-37 and ART-5 are proteins of unknown function represented in the Expressed,Sequence Tag (EST) database, whereas ART-27 is identical to ubiquitously expressed transcript (UXT), a recently identified open reading frame on the X chromosome (Xp11.23-11.22) that encodes a putative ˜18 kDa protein of unknown function (Schroer et al., 1999).

Intermediate strength interactors include ART-6, an EST, and ART-15, which is identical to ATBF1a, a transcription factor containing multiple zinc finger and homeodomain motifs that was isolated in a screen for proteins that bind to the alpha-fetoprotein enhancer (Visakorpi et al, 1995b). Weak interactors include ART-9, which corresponds to ZNF160 (Halford et al., 1995), a zinc finger containing protein of unknown function, and ART-2 and ART-3, which are present in the EST database.

ART Interaction Specificity

To analyze the specificity of ART interaction, the capacity of the strongest androgen receptor N-terminus-interacting factors to associate with a panel of transcriptional regulatory proteins in the modified yeast two-hybrid assay was examined. ART-5, ART-27, and ART-37 were tested for interaction with Sp1A (SP1₈₃₋₂₆₂), Sp1B (Sp1₂₆₃₋₅₂₄) the cyclic AMP response element binding protein (CREB₃₋₂₉₆), TBP-associated factor 130 (TAF_{II}130₂₇₀₋₇₀₀), the glucocorticoid receptor AF1 (GR₁₀₇₋₂₃₇), and the steroid receptor coactivator-1 (SRC-1₃₇₄₋₈₀₀).

FIG. 1B shows the specificity of ART-37, ART-27 and ART-5 with androgen receptor (AR) N-terminus (18-500), androgen receptor ligand-binding domain (579-901) and other transcriptional regulatory factors was analyzed using the modified yeast two-hybrid assay. The strength of interaction was determined by a qualitative plate beta-galactosidase assay after a 24 hour incubation on galactose X-gal plates at 30° C. Strong interactions (+) represent blue colonies, and (−) represents no interactions above background “vector only” (white colony).

From FIG. 1B, it can be seen that ART-5 interacts exclusively with the androgen receptor N-terminus, whereas ART-27 interacts with the androgen receptor (AR) and glucocorticoid receptor (GR) N-termini, as well as with Sp1 and with TAF_{II}130, but not with SRC-1 or CREB. No interaction between the androgen receptor ligand binding domain and ART-5, ART-27, or ART-37 was observed in either the absence or presence of hormone. In contrast, ART-37 is relatively promiscuous, interacting with virtually all of the transcriptional regulators examined. These results indicate that ART-5 interacts rather specifically with the androgen receptor N-terminus, ART-27 displays less selectivity, interacting with the androgen receptor N-terminus and with certain other transcriptional regulatory factors including TAF_{II}130, whereas ART-37 is unable to discriminate among the factors examined.

ART and mRNA Expression

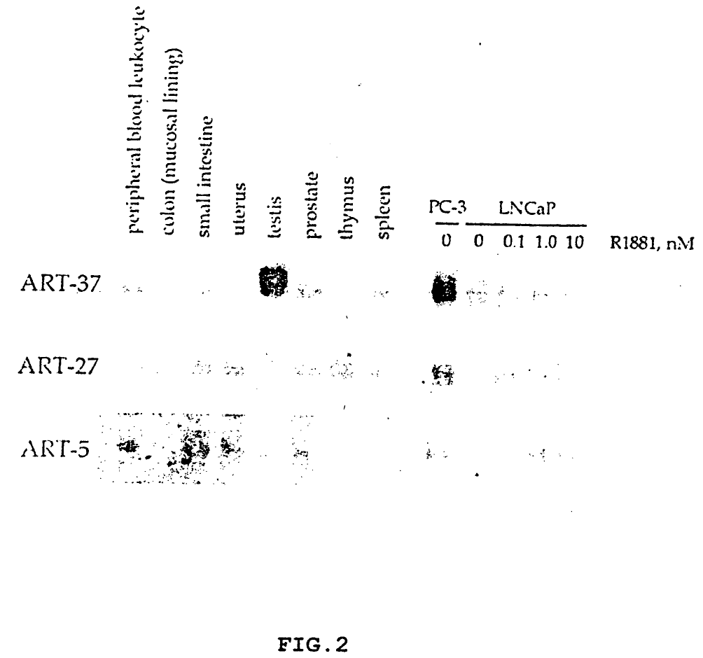

Using ART-5, ART-27, and ART-37, Northern blot analysis was performed on mRNA isolated from androgen-independent (PC-3) and androgen-dependent (LNCaP) prostate cancer cells, either untreated or stimulated for 72 hours with the synthetic androgen R1881 at the concentrations indicated in FIG. 2 (right panel). In this analysis, equal amounts of RNA were separated on denaturing formaldehyde-agarose gels, transferred to Duralon nylon membrane, and hybridized to ³²P-labeled cDNA probes corresponding to ART-37, ART-27 and ART-5 (right panel). Equal loading for each lane was determined by ethidium bromide staining of the 28S rRNA (not shown). A human multiple tissue northern blot (Clontech: MTN Blot IV) containing 2 micrograms of poly A+ mRNA from the tissues indicated was hybridized with ³²P-labeled probes corresponding to ART-37, ART-27, and ART-5 (left panel). It was found that ART-37 mRNA (˜1.2-kb) was highly expressed in PC-3 cells relative to LNCaP cells, while ART-5 (˜1.4 kb) steady state mRNA concentration was similar in both cell types.

In examining whether androgens regulate ART expression in LNCaP cells, it was found that ART-27 and ART-4 showed a small increase in steady state mRNA expression in LNCaP cells in response to increasing concentrations of androgen. ART-37 RNA levels were however not affected.

As shown in FIG. 2 (left panel), multiple human tissue blots were probed for ART expression. ART-5, ART-27 and ART-37 appear to be widely expressed in human tissues, including normal human prostate tissue. ART-27 mRNA appears uniformly expressed in the tissues examined. In contrast, ART-37 and ART-5 mRNA expression varies among tissues, with the highest level of ART-37 mRNA in thevtestis and lowest in the thymus. ART 5 expression was found to be greatest in the small intestine and lowest in the colon. These results indicate that ART-5, ART-27 and ART-37 are expressed in a variety of normal human tissues and display differential patterns of expression in prostate cancer cell lines.

ART-27 Localizes Predominantly to the Nucleus

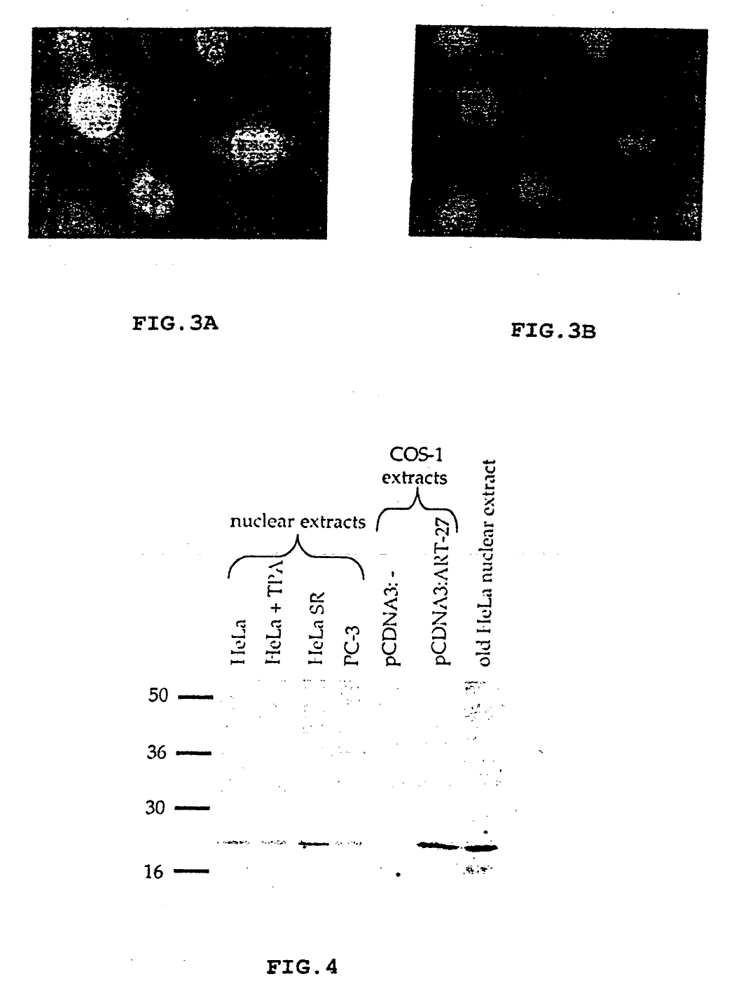

Since the ART-27 cDNA clone isolated in the screens contains the complete coding sequence, a mammalian expression vector was created for the full-length ART-27 containing a HA-epitope tag at its N-terminus. HeLa cells were transiently transfected with an HA-ART-27 construct, fixed, permeabilized, and incubated with an anti-HA primary antibody, a corresponding rhodamine-conjugated secondary antibody, and the DNA in the nucleus was stained with Hoechst dye H334211. The rhodamine and Hoechst fluorescent signals were visualized using a Zeiss Axioplan 2 fluorescence microscope. No signal was observed above background when the primary antibody was omitted and the cells were stained with the rhodamine-conjugated secondary antibody (not shown). ART-27 was found to localize predominantly to the nucleus, although some diffuse staining was apparent in the cytoplasm of cells expressing high levels of the protein, as shown in FIGS. 3A and 3B. This predominant nuclear distribution of ART-27 is consistent with its role as a transcriptional regulatory protein.

FIG. 4 shows immunoblotting with nuclear extracts derived from different indicated cell types using an ART-27-specific polyclonal antibody. An affinity purified polyclonal antibody raised against the C-terminus of human ART-27 was used to probe nuclear extracts from HeLa and PC3 cells. An ART-27 immunoreactive band of apparent MW ˜18 kDa was observed to co-migrate with ART-27 expressed in COS-1 cells.

ART-27 Interacts with Androgen Receptor in vitro

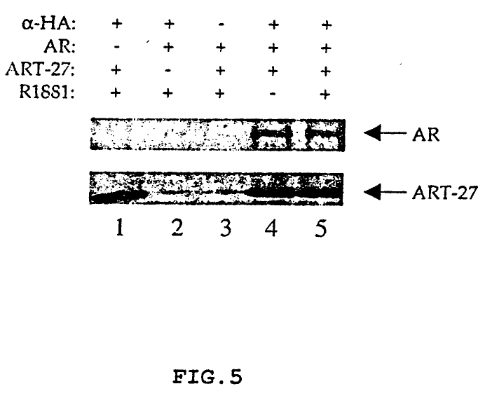

The ability of ART-27 and AR to interact was also tested in vitro. Full length androgen receptor and HA-ART-27 were expressed in a coupled transcription/translation system in the presence of ³⁵S methionine, in the absence or presence of 100 nM R1881, as indicated in FIG. 5, and immunoprecipitated with an antibody against the epitope on ART-27 HA. Bound proteins were collected on Protein A Sepharose beads, washed, eluted, and resolved by SDS-PAGE and visualized by autoradiography. In this co-immunoprecipitation assay, in vitro translated full length HA-ART-27 bound in vitro synthesized androgen receptor in the presence and absence of the hormone, as shown in FIG. 5. Androgen receptor was not immunoprecipitated with the HA-antibody in the absence of coexpressed AH-ART-27. These results substantiate the androgen receptor-ART-27 interaction observed in the yeast two-hybrid system.

Domains Involved in Androgen Receptor-ART-27 Interaction.

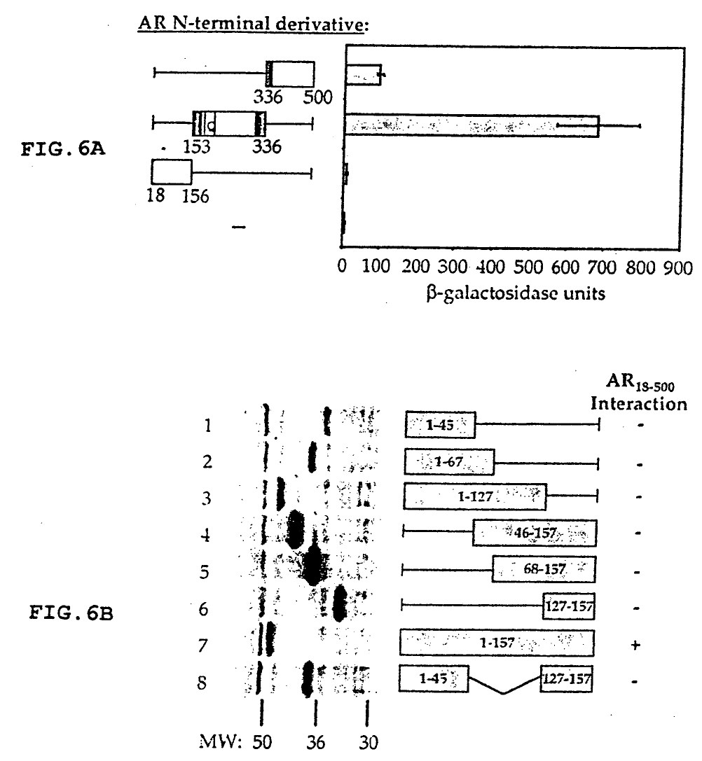

To locate the region(s) within the androgen receptor N-terminus that interacts with ART-27, AR₈₅₋₅₀₀ was divided into three subdomains: AR₁₈₋₁₅₆, AR₁₅₃₋₃₃₆, and AR₃₃₆₋₅₀₀, and the relative affinity of ART-27 for these subdomains was assessed using the modified yeast interaction-trap assay (FIG. 6A). The dark gray boxes in FIG. 6A represent AF-1a and AF-1b, and the light gray box denotes the glutamine (Q) repeat region. Data represent the mean of triplicate data points normalized to cell number. It was found that ART-27 has the highest affinity for the AR₁₅₃₋₃₃₆ region, a region encompassing all of AF-1a (residues 154-167) and a small part of the AF-1b residues (295-259). A weak interaction between ART-27 and the AR₃₃₆₋₅₀₀ subdomain was also observed, whereas no interaction was detected between ART-27 and AR₁₈₋₁₅₆. Immunoblot analysis of the AR₁₈₋₁₅₆, AR₁₅₃₋₃₃₆, and AR₃₃₆₋₅₀₀ derivatives indicated that they are expressed at similar levels (not shown). These findings suggest that the AR₁₅₃₋₃₃₆ region is the primary androgen receptor N-terminal interaction site for ART-27.

In an attempt to localize the region of ART-27 that interacts with the androgen receptor N-terminus, a series of ART-27 B and C-terminal derivatives were created. ART-27 derivatives containing amino acids 1-45, 1-67, 1-127, 46-157, 68-157, 127-157, 1-157, and 1-45/127-157 were expressed as fusion proteins with LexA. These derivatives were tested for their ability to interact with the androgen receptor N-terminus (AR₁₈₋₅₀₀). The strength of interaction was determined by a qualitative plate beta-galactosidase assay after a 24 hour incubation on galactose X-gal plates at 30° C. Strong interactions (+) represent blue colonies, and (−) represents no interactions above background “vector only” control (white colony). The left panel of FIG. 6B shows an immunoblot of the ART-27 derivatives expressed in yeast and probed with an antibody against the LexA moiety common to all ART-27 truncations. Surprisingly, none of the N- or C-terminal deletion derivatives interacted with AR₁₈₋₅₀₀ (FIG. 6B), even though all of the ART-27 derivatives were expressed (FIG. 5B, left panel). This result suggests that either ART-27 required multiple contacts for interaction with the androgen receptor N-terminus or that the entire protein is involved in configuring a functional AR interacting surface.

ART-27 Enhances Androgen Receptor Ligand-dependent Transcriptional Activation in Mammalian Cells

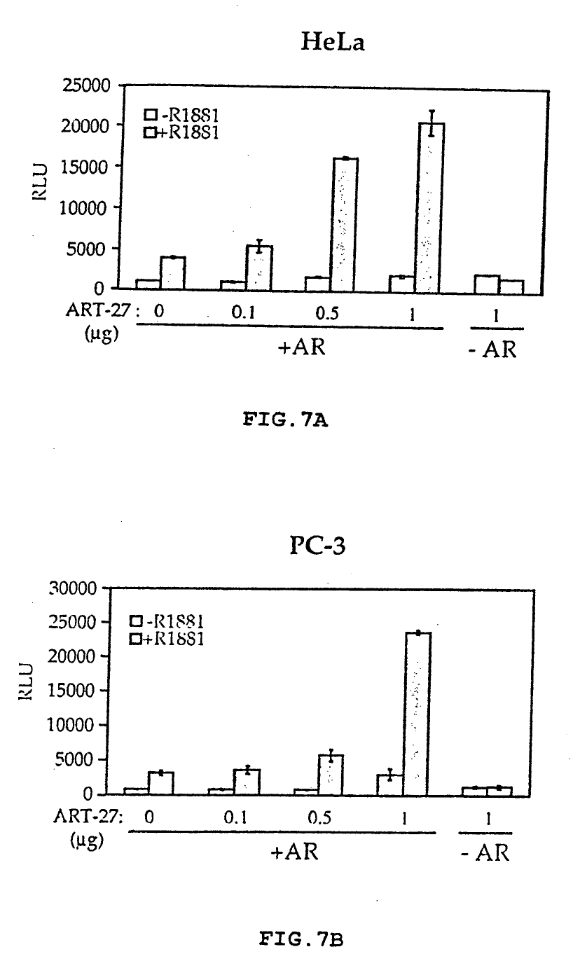

Since ART-27 interacts with the androgen receptor N-terminus, it was anticipated that ART-27 would play a role in androgen receptor-dependent transcriptional regulation. To establish whether overexpression of ART-27 affects androgen receptor transcriptional activities, androgen receptor deficient HeLa cells (FIG. 7A) and PC-3 cells (FIG. 7B), both AR deficient, were transfected with a constant amount of full length androgen receptor and increasing concentrations of an expression vector encoding a full length HA-tagged ART-27 (2 micrograms per dish) along with an AR-responsive luciferase reporter gene and CMV-beta-galactosidase (0.5 microgram per dish) as an internal standard for transfection efficiency. Adding empty expression vector equalized the total amount of DNA per dish. The cells were treated with the 100 nM R1881 (shaded bars) or the ethanol vehicle (white bars) for twelve hours and androgen receptor transcriptional activation was assayed, normalized to beta-galactosidase activity, and expressed as relative luminescence units (RLU). The average of three independent experiments is shown with standard error.

As shown in FIG. 7A, hormone-dependent androgen receptor transcriptional activation was increased in a dose-dependent manner when ART-27 is overexpressed. This effect was dependent on androgen receptor, since in the absence of androgen receptor, ART-27 did not influence reporter gene activity (FIGS. 7A and 7B). To ensure that this enhanced transcriptional activity was not the result of increased androgen receptor protein production, protein expression was monitored, and it was found that androgen receptor levels were not affected by ART-27 coexpression (not shown).

The effect of ART-27 on androgen receptor was not restricted to a single cell type, since overexpression of ART-27 in PC-3 and COS-1 cells also resulted in a dose-dependent increase in androgen receptor transcriptional activity (FIG. 7B and not shown). Androgen receptor ligand-independent transcriptional activation was also increased when ART-27 is overexpressed at the highest concentrations in both PC-3 and HeLa cells. Thus, ART-27 expression enhances the androgen receptor-dependent transcriptional response, both ligand-dependent and ligand-independent, which suggests that ART-27 can act as a regulator of androgen receptor transcriptional activity in mammalian cells.

It was next determined whether an ART-27 derivative lacking the androgen receptor-interacting region and incapable of interacting with androgen receptor was capable of affecting androgen receptor-mediated transcriptional activity. HeLa cells were transfected with androgen receptor, along with an androgen receptor-responsive luciferase reporter gene and either an empty expression vector, full length ART (1-157), or a C-terminal deletion derivative of ART-27 (1-127) that was unable to interact with the androgen receptor N-terminus in the two-hybrid assay. Androgen receptor activity was determined in the presence of 100 nM R1881 as described for FIGS. 7A and 7B. The data represent the mean of duplicate data points normalized to beta-galactosidase units.

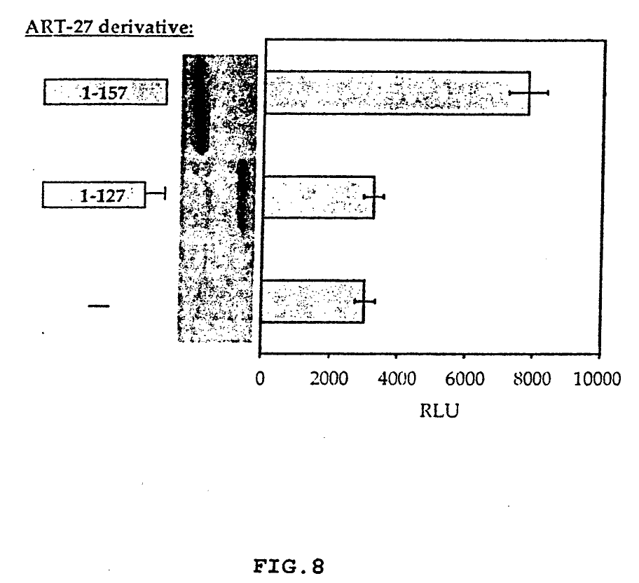

As shown in FIG. 8, whereas full length ART-27 is capable of enhancing androgen receptor transcriptional activity, ART-27₁₋₁₂₇ is not, even though they are expressed to comparable levels. These results indicate that the enhanced androgen receptor transactivation observed upon ART-27 overexpression is dependent upon an androgen receptor-ART-27 interaction.

Enhanced Androgen Receptor-dependent Transcriptional Activation by ART-27 is Mediated Through a Distinct Receptor N-terminal Domain

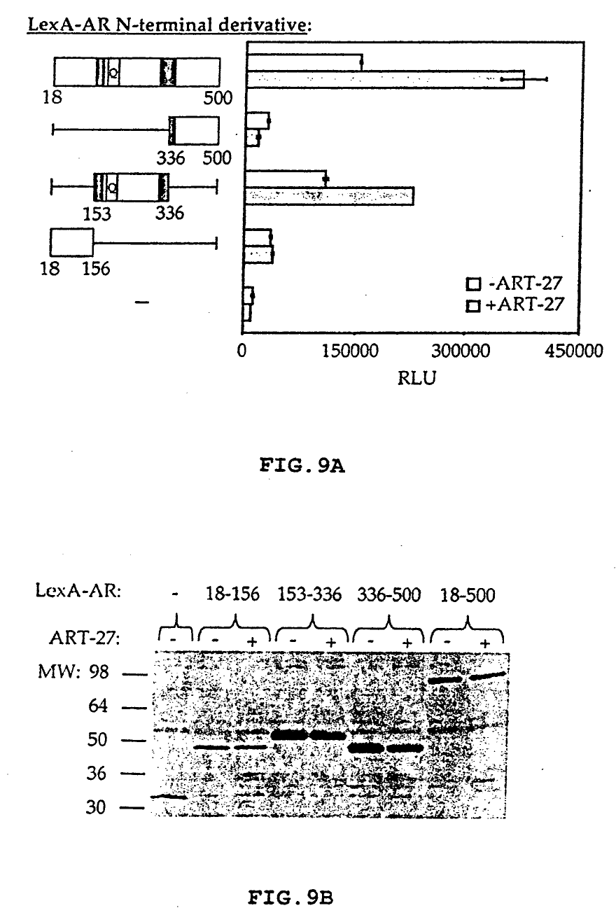

Because ART-27 interacts most strongly with the androgen receptor subdomain spanning amino acids 153-336 (FIG. 6A), it is expected that it would affect the transcriptional activation potential of this androgen receptor subdomain. To determine if ART-27 could affect the function of the different androgen receptor subdomains, androgen receptor N-terminal derivatives containing amino acids 18-156, 153-336, 336-500, and 18-500 were expressed as fusion proteins with the LexA DNA binding domain. HeLa cells were transiently transfected with the LexA:AR N-terminal derivatives and either an empty expression vector (white bars in FIG. 9A) or full length HA-ART-27 (shaded bars) along with an LexA responsive-luciferase reporter gene. Androgen receptor activity was determined as in FIGS. 7A and 7B in the presence or absence of ART-27. In the absence of ART-27 coexpression, all four subdomains of the androgen receptor N-terminus were capable of activating transcription of the LexA-luciferase reporter gene to varying degrees, as shown in FIG. 9A. Importantly, overexpression of ART-27 enhances the transcriptional activity to two androgen receptor derivatives containing the ART-27 interaction regions, LexA-AR₁₅₃₋₃₃₆, and Lex-AR₁₈₋₅₀₀, but not the transcriptional activity of the derivatives lacking the primary ART-27 interaction regions, LexA-AR₁₈₋₁₅₆ and LexA-AR₃₃₆₋₅₀₀. In fact, transcriptional activation of the LexA-AR₃₃₆₋₅₀₀ derivative was slightly reduced by ART-27 overexpression, suggesting that ART-27 may interact with and sequester a factor responsible for androgen receptor transactivation via the 336-500 subdomain.

To verify that the expression of the LexA:AR derivatives was not affected by ART-27 overexpression, a parallel set of transfections were analyzed by immunoblotting with a polyclonal antibody to LexA. As shown in FIG. 9B, expression of these chimeras is unaffected by coexpression of ART-27 in HeLa cells. These results indicate that the enhanced androgen receptor transcriptional activation observed upon ART-27 overexpression depends upon the ART-27-androgen receptor-interacting portion.

ART-27 Overexpression Affects Androgen Receptor Ligand Potency

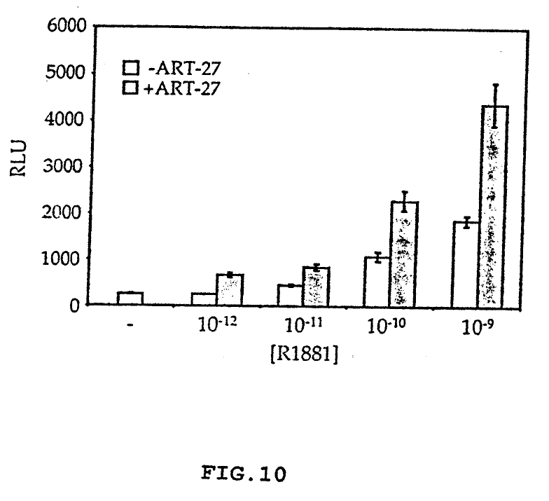

It has recently been shown that overexpression of steroid receptor coactivators and corepressors can influence the dose response curve, effectively lowering or raising the threshold of hormone necessary to achieve transcriptional activation (Szapary et al., 1999). To examine whether ART-27 overexpression shifts the dose response curve of androgen receptor to androgen, HeLa cells were transfected with a constant amount of androgen receptor (0.2 microgram/dish), empty expression vector (white bars in FIG. 10) or HA-ART-27 (1 microgram/dish) (shaded bars in FIG. 10) and an androgen receptor responsive reporter gene (0.1 micrograms per dish). The cells were treated with the ethanol vehicle (−) or with the indicated amounts (FIG. 10) of R1881 for twelve hours and androgen receptor transcriptional activation was assayed as for FIGS. 7A and 7B. The (−) lane represents cells transfected with an expression vector encoding LexA alone.

The results shown in FIG. 10 demonstrate that the androgen receptor transcriptional response observed in the absence of ART-27 is achieved at a lower ligand concentration in the presence of ART-27. For example, the androgen receptor transcriptional response observed at 10⁻⁹ M R1881 in the absence of ART-27 is achieved at a ten-fold lower ligand concentration (10⁻¹ M R1881) in the presence of ART-27 (FIG. 10). Thus, overexpression of ART-27 not only affects ligand efficacy (maximal activation levels at saturating hormone concentrations), but also ligand potency (responding to lower concentration of androgen), suggesting that ART-27 plays important roles in determining the sensitivity and activity of androgen receptor to androgen in target cells. ART-27 enhances GR and ER alpha-dependent transcriptional activation

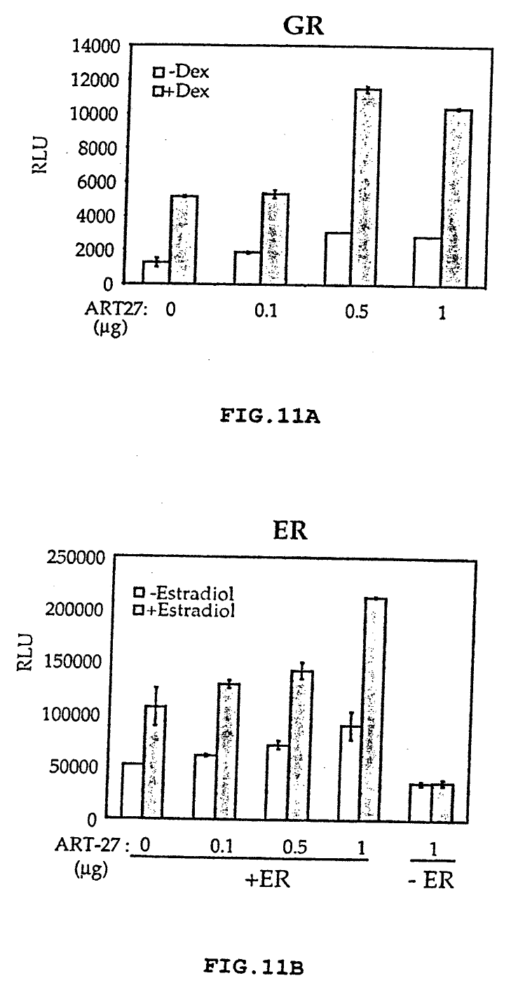

HeLa cells were transfected with expression plamids for (A) human glucocorticoid receptor (GR) (FIG. 11A) or the human estrogen receptor alpha (+ER) (FIG. 11B) and ART-27 at the indicated amounts along with a GRE or ERE-Luciferase reporter construct (2 μg/dish) and CMV-β-galactosidase (0.5 μg/dish) as an internal standard for transfection efficiency. Adding empty expression vector equalized the total amount of DNA per dish. Cells were treated with 100 nM Dexamtheasone (Dex) or 17-b-estradiol (Estradiol) (shaded bars) or the ethanol vehicle (white bars) for 12 hr and receptor transcriptional activation was assayed, normalized to β-galactosidase activity and expressed as relative luminescence units (RLU). The average of three independent experiments is shown with standard error.

ART-27 Enhances ER Alpha, but not ER Beta-dependent Transcriptional Activation

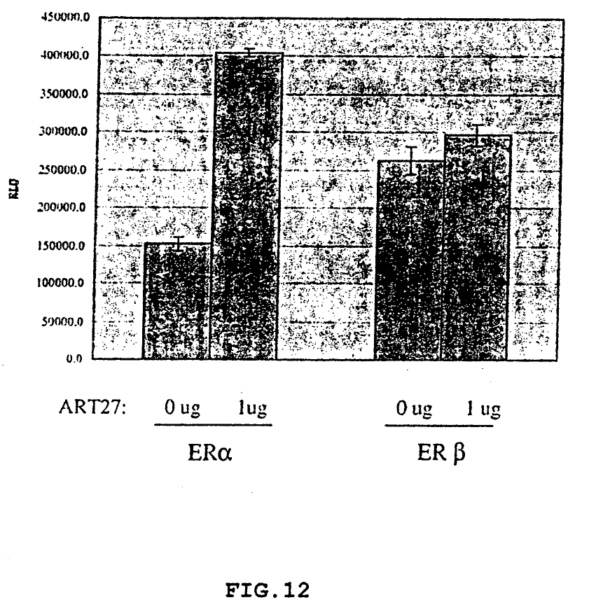

In FIG. 12, U2OS cells were transfected with expression plasmids for human estrogen receptor alpha (+ER α) or the human estrogen receptor beta (+ER β) and ART-27 at the indicated amounts along with an ERE-Luciferase reporter construct and CMV-β-galactosidase as an internal standard for transfection efficiency. Adding empty expression vector equalized the total amount of DNA per dish. Cells were treated with 100 nM 17-β-estradiol for 12 hours and receptor transcriptional activation was assayed, normalized to β-galactosidase activity and expressed as relative luminescence units (RLU). It can be seen that ER alpha interacts with ART-27 in the yeast two hybrid system, whereas ER beta does not. Therefore, the effect of ART-27 on ER transcriptional activation correlates with its ability to interact.

ART-27 expression in Matched Normal and Tumor Tissues

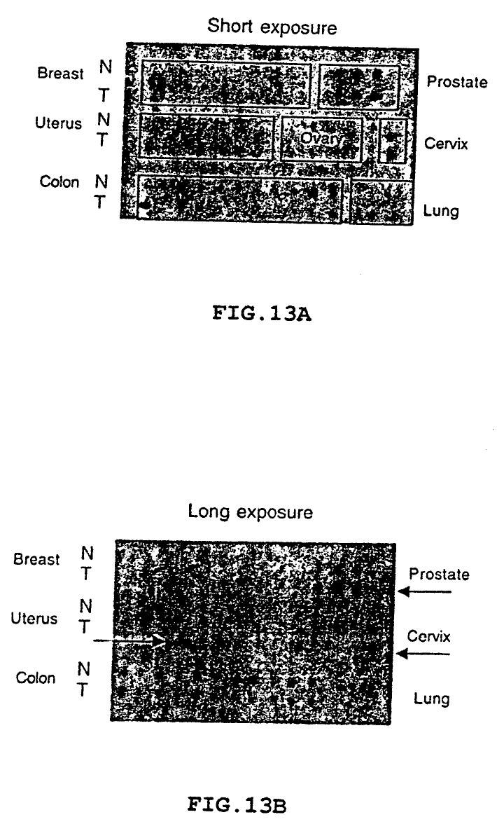

Matched Normal and Tumor Expression Array (Clontech) was hybridized with ART-27 cDNA (FIGS. 13A and 13B) mRNAs from matched normal (N) and tumor (T) specimens from the indicated tissues were reversed transcribed into cDNA and arrayed onto a filter. FIG. 13A is 4-hour exposure (short) and FIG. 13B is a 16 hour exposure (long) of the filter. It can be seen that ART-27 mRNA is most abundant in normal prostate and is overexpressed in at least one prostate tumor, the single cervical tumor sample and several uterine tumor specimens. Expression of ART-27 is low in normal and tumor breast, ovary and lung samples.

Regulation of ART-27 Protein Expression in a Rat Androgen-depletion model

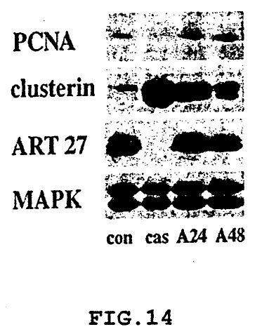

The endogenous expression of ART-27 was also examined in a rat androgen-depletion model. Rats were castrated to cause withdrawal of testicular androgens and atrophy of the prostate gland. Later, androgens were then re-administered resulting in cellular proliferation and recapitulation of the prostate. In this experiment (FIG. 14), prostates were dissected from rats and lysates were made under the following conditions; untreated (con), 96-hours post-castration (cas), 96 hours post-castration plus 48 hours treatment with androgens (A24), and 96 hours post-castration plus 72 hours treatment with androgens (A48). The lysates were then normalized for protein expression and used for Western blot analysis. The filters were incubated with antibodies against proliferating cell nuclear antigen (PCNA—a marker for cellular proliferation), clusterin (a marker for apoptosis), ART-27, and MAP kinase (MAPK) as an internal control for protein loading of the gel. As expected, PCNA expression is abolished following castration, and upregulated upon re-administration of androgens when prostate cells are once again proliferating. The expression of clusterin, which is also known as testosterone repressed prostate message-2 (TRMP-2), is normally low, and greatly upregulated following castration.

The results show that while MAPK is represented approximately equally in all lanes, ART-27 protein is dramatically reduced following androgen withdrawal (cas), but is abundant when androgens are available (cas, A24 and A48). Thus, ART-27 is present in prostate tissue and the results suggest that it is regulated by androgens, consistent with the hypothesis that ART-27 plays a role in AR-mediated cell growth and transcription.

ART-27 Expression in Human Prostate by Immunohistochemistry

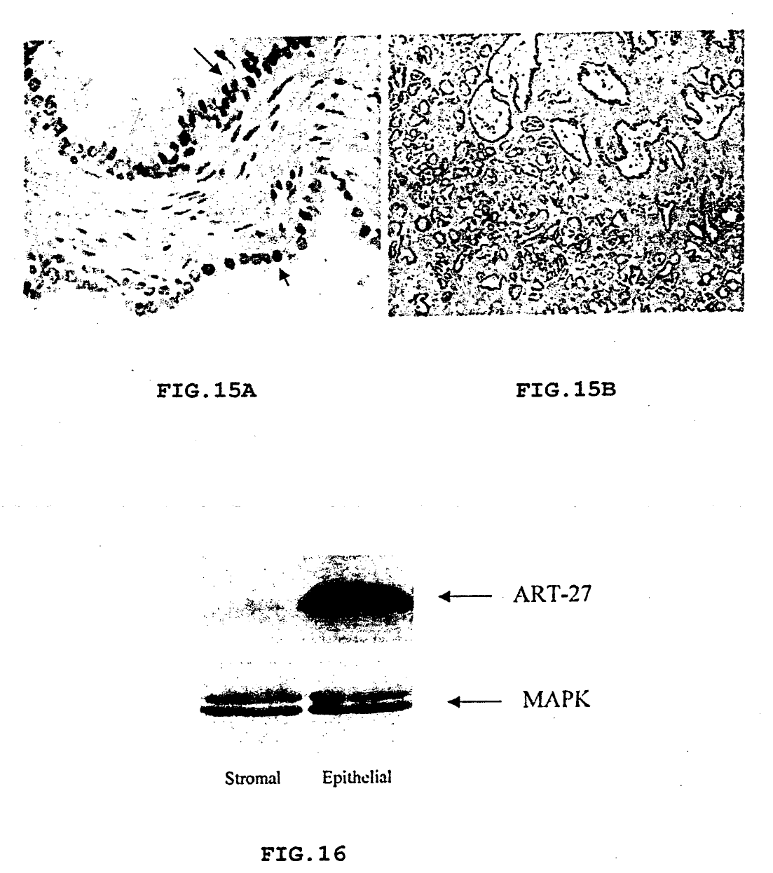

Examination of ART-27 immunoreactivity on archival formalin fixed paraffin sections shows strong epithelial cell staining in human prostate tissue. FIG. 15A shows immunohistochemical analysis of paraffin embedded human prostate tissue treated with affinity purified ART-27 antibody (400× magnification). Arrows indicate antibody reactivity with nuclei of epithelial cells. Stromal cells, which are oriented horizontally to the two epithelial cell layers are visible in the central portion of FIG. 15A and do not appear to express ART-27. FIG. 15B shows staining in paraffin embedded archival tissue from a prostate carcinoma (2× magnification). The upper right diagonal field is “normal” while the lower left diagonal field is carcinoma as indicated in that the nepotistic glands have infiltrative growth and aberrant prostatic architecture. The staining is seen in both basal and luminal epithelial cells and there is little, if any staining in stromal tissue. Importantly, since androgen receptor expression also occurs in the prostate epithelial cells, ART-27 is found to be expressed in androgen receptor positive cells in the prostate.

Immunoblot Analysis of ART-27 Expression in Primary Human Prostate Cells

To further characterize the tissue specific expression of ART-27, expression using explant cultures from primary human epithelial and stromal cells was examined. Protein extracts were made from primary human stromal or epithelial cell explant cultures. Proteins were run on an acrylamide gel, transferred to nitrocellulose, and incubated with antibodies against either ART-27 or MAPK (as an internal loading control). Consistent with in vivo results from-immunohistochemistry, ART-27 is found to be highly expressed in epithelial cells, and expressed at low levels, if at all, in stromal cells (FIG. 16).

### Discussion

ART-27 has thus been identified as a protein that interacts with the androgen receptor N-terminal subdomain spanning amino acids 153-336, a region that encompasses the whole of AF-1a (154-167) and part of AF-1b (295-459), and enhances androgen receptor transcriptional activation when overexpressed in mammalian cells. The ability of ART-27 to affect androgen receptor transcription activation depends upon the ART-27 androgen receptor-interacting region, since only the androgen receptor N-terminal derivatives containing the interaction domain are enhanced by ART-27 coexpression. Thus, ART-27 represents an androgen receptor N-terminus-associated coactivator.

ART-27 was originally identified in a screen for novel genes that map to the human Xp11 locus, a region previously shown to contain an abundance of disease loci, which led to the identification of a novel ubiquitously expressed transcript (UXT)(Schroer et al., 1999). The results obtained herein suggest that ART-27/UXT functions as a transcriptional coactivator, increasing androgen receptor-dependent transcriptional activation through direct binding to the androgen receptor N-terminus. Interestingly, ART-27 and androgen receptor reside in an amplicon found in a subset of hormone-refractory prostate cancers, suggesting that ART-27 may play a role in androgen receptor-dependent prostate tumorigenesis (Visakorpi et al., 1995a and 1995b). It may be possible that progression to hormone-refractory prostate cancer may occur through the amplification of the androgen receptor gene and its cognate N-terminal coactivator, ART-27, resulting in greater sensitivity to low levels of circulating androgens. Consistent with this hypothesis, ART-27 overexpression appears to affect androgen receptor ligand potency and lowers the threshold concentration or androgen required for full androgen receptor-dependent transcriptional activation.

One potential explanation for why the entire ART-27 protein is required for interaction with androgen receptor is that ART-27 may associate with the androgen receptor N-terminus through multiple low affinity interactions, and removal of any one of these contacts renders ART-27 incapable of association. Alternatively, the complete ART-27 may be involved in configuring a functional protein and its integrity may be compromised upon deletion of any region. Secondary structure predictions for ART-27 suggest that it is composed of four contiguous alpha-helices. Whether each helix represents an independent interaction surface for androgen receptor or these helices function together to coordinate the tertiary structure of the protein in vivo will require a detailed structure-function analysis of ART-27.

The mechanism by which ART-27 affects androgen receptor-mediated transcriptional activation has not yet been defined. ART-27 is a comparatively small protein with a predicted molecular mass of ˜18 kDa, and has little transcriptional activation ability when tethered to DNA in yeast, suggesting that it does not initiate transcription directly. Since many of the transcriptional regulatory cofactors have recently been identified as components of multiprotein complexes, it is possible that ART-27 may represent a subunit of a previously characterized (e.g., DRIP/TRAP/ARC or TFIID),or novel multi protein coactivator complex (Glass et al., 2000). Although many of the proteins in the DRIP/TRAP/ARC complex have been identified, several low molecular weight species have yet to be analyzed, which may include ART-27. It is interesting to note that ART-27 interacts with TAFI,130 in the yeast two-hybrid assay, suggesting that ART-27 communicates with at least one member for the TFIID complex. Preliminary studies also suggest that TAF_{II}130 interacts with and increases androgen receptor transcriptional activation via the androgen receptor N-terminal subregion 336-500. Since ART-27 and TAF_{II}130 interact in the system shown in FIG. 1B, it is believed that the reduced transcriptional activation of the LexA-AR₃₃₆₋₅₀₀ derivative upon ART-27 overexpression (FIG. 9) represents the sequestration of TAF_{II}130 by ART-27. Alternatively, since ART-27 also interacts weakly with AR₃₃₆₋₅₀₀, it may associate with this domain in a non-productive fashion and inhibit its function.

Thus, the androgen receptor N-terminus appears to be a multifaceted platform capable of interacting with a variety of transcriptional regulatory proteins, including ART-27, which collaborate with to regulate gene- and tissue-specific responses to androgen receptor. Consistent with this notion, the coactivators SRC-1, GRIP-1 and CBP have recently been shown to interact with the androgen receptor N-terminus and modulate its activity (Bevan et al., 1999; Alen et al., 1999; Ikonen et al., 1997 and Ma et al., 1999) ART-27 and other ARTs represent an important new class of prognostic markers and therapeutic targets for prostate cancer and other androgen receptor-dependent maladies, including benign prostate hyperplasia and androgen-dependent hair loss.

Having now fully described this invention, it will be appreciated that by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional method steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Alen et al., “The androgen receptor amino-terminal domain plays a key role in p160 coactivator-stimulated gene transcription”, Mol Cell Biol, 19:6085-97 (1999)

Baker, A. R., McDonnell, D. P., Hughes, M., Crisp, T. M., Mangelsdorf, D. J., Haussler, M. R., Pike, J. W., Shine, J. and bO'Malley, B. W., “Cloning and expression of full_length CDNA encoding human vitamin D receptor”, Proc. Natl. Acad. Sci. U.S.A. 85 (10), 3294-3298 (1988)

Bevan et al., “The AF1 and AF2 domains of the androgen receptor interact with distinct regions of SRC1”, Mol Cell Biol, 19:8383-92 (1999)

Brinkmann et al., “The human androgen receptor structure/function relationship in normal and pathological situations”, J Steroid Biochem Mol Biol, 41:361-8 (1992)

Chamberlain et al., “Delineation of two distinct type 1 activation functions in the androgen receptor amino-terminal domain”, J Biol Chemi, 271:26772-8 (1996)

Chang et al., “Androgen receptor: an overview”, Crit Rev Eukaryot Gene Expr, 5:97-125 (1995)

Chen et al., “Nuclear receptor coactivator ACTR is a novel histone acetyltransferase and forms a multimeric activation complex with P/CAF and CBP/p300”, Cell, 90:569-80 (1997)

Cleutjens et al., “Both androgen receptor and glucocorticoid receptor are able to induce prostate-specific antigen expression, but differ in their growth-stimulating properties of LNCaP cells”, Endocrinology, 138:5293-300 (1997)

Dorkin et al., “The molecular pathology of urological malignancies”, J Pathol, 183:380-7 (1997)

Dorkin et al., “Basic science aspects of prostate cancer”, Semin Cancer Biol, 8:21-7 (1997)

Duina et al., “A cyclophilin function in Hsp90-dependent signal transduction”, Science, 274:1713-5 (1996)

Elbrecht, A., Chen, Y., Cullinan, C. A., Hayes, N., Leibowitz, Md., Moller, D. E. and Berger, J., “Molecular cloning, expression and characterization of human peroxisome proliferator activated receptors gamma 1 and gamma 2”, Biochem. Biophys. Res. Commun. 224 (2), 431-437 (1996)

Eshhar et al., “Chimeric T cell receptor which incorporates the anti-tumour specificity of a monoclonal antibody with the cytolytic activity of T cells: a model system for immunotherapeutical approach”, Br. J. Cancer Suppl., 10:27-9 (1990)

Fang et al., “Hsp90 regulates androgen receptor hormone binding affinity in vivo”, J Biol Chem, 271:28697-702 (1996)

Fang et al., “SBA1 encodes a yeast hsp90 cochaperone that is homologous to vertebrate p23 proteins”, Mol Cell Biol, 18:3727-34 (1998)

Flanagan, “Antisense comes of age”, Cancer Metastasis Rev. 17, p. 169-76, (1998)

Garabedian et al., “Genetic dissection of the signaling domain of a mammalian steroid receptor in yeast”, Mol Biol Cell, 3:1245-57 (1992)

Glass et al., “The coregulator exchange in transcriptional functions of nuclear receptors”, Genes Dev, 14:121-41 (2000)

Gerster et al., “Quantitative analysis of modified antisense oligonucleotides in biological fluids using cationic nanoparticles for solid-phase extraction”, Anal. Biochem. 262, p. 177-84, (1998)

Giguere, V., Ong, E. S., Segui, P. and Evans, R. M., “Identification of a receptor for the morphogen retinoic acid”, Nature 330 (6149), 624-629 (1987)

Gordon et al., “A cell-specific and selective effect on transactivation by the androgen receptor”, Exp Cell Res, 21:368-77 (1995)

Green, S., Walter, P., Kumar, V., Krust, A., Bornert, J. M., Argos, P. and Chambon, P., “Human oestrogen receptor cDNA: sequence, expression and homology to v-erb-A”, Nature 320 (6058), 134-139 (1986)

Gross et al., Expression of immunoglobulin-T-cell receptor chimeric molecules as functional receptors with antibody-type specificity”, Proc. Natl. Acad. Sci. USA, 86:10024-8 (1989)

Guinot et al., “Antisense oligonucleotides: a new therapeutic approach” Pathol. Biol. (Paris) 46, p. 347-54, (1998)

Hakimi et al., “Androgen-receptor gene structure and function in prostate cancer”, World J Urol, 14:329-37 (1996)

Halford et al., “A novel C2H2 zinc-finger protein gene (ZNF160) maps to human chromosome 19q13.3-q13.4”, Genomics, 25:322-3 (1995)

Harlow, E., Williamson, N. M., Ralston, R., Helfman, D. M. and Adams, T. E., “Molecular cloning and in vitro expression of a cDNA clone for human cellular tumor antigen p53”, Mol. Cell. Biol. 5 (7), 1601-1610 (1985)

Hirose, T., Smith, R. J. and Jetten, A. M. “ROR gamma: the third member of ROR/RZR orphan receptor subfamily that is highly expressed in skeletal muscle”, Biochemi. Biophys. Res. Commun. 205 (3), 1976-1983 (1994)

Hittelman et al., “Differential regulation of glucocorticoid receptor transcriptional activation via AF-1-associated proteins”, Embo J, 18:5380-5388 (1999)

Hong et al., “GRIP1, a transcriptional coactivator for the AF-2 transactivation domain of steroid, thyroid, retinoid, and vitamin D receptors”, Mol Cell Biol, 17:2735-44 (1997)

Hsiao et al., “Isolation and characterization of ARA160 as the first androgen receptor N-terminal-associated coactivator in human prostate cells”, J Biol Chem, 274:22373-9 (1999)

Ikonen et al., “Interaction between the amino- and carboxyl-terminal regions of the rat androgen receptor modulates transcriptional activity and is influenced by nuclear receptor coactivators”, J. Biol Chem, 272:29821-8 (1997)

Jenster et al., “Domains of the human androgen receptor involved in steroid binding, transcriptional activation, and subcellular localization”, Mol Endocrinol, 5:1396-404 (1991)

Jenster et al., “Functional domains of the human androgen receptor”, J Steroid Biochem Mol Biol, 41:671-5 (1992)

Jenster, “The role of the androgen receptor in the development and progression of prostate cancer”, Semin Oncol, 26:407-21 (1999)

Kang et al., “Cloning and characterization of human prostate coactivator ARA54, a novel protein that associates with the androgen receptor”, J Biol Chem, 274:8570-6 (1999)

Kastner, P., Krust, A., Turcotte, B., Stropp, U., Tora, L., Gronemeyer, H. and Chambon, P., “Two distinct estrogen-regulated promoters generate transcripts encoding the forms A and B”, EMBO J. 9 (5), 1603-1614 (1990)

Knoblauch et al., “Role for Hsp90-associated cochaperone p23 in estrogen receptor signal transduction”, Mol Cell Biol, 19:3748-59 (1999)

Kondo et al., “Modulation of apoptosis by endogenous Bcl-xL expression in MKN-45 human gastric cancer Cells”, Oncogene 17, p. 2585-91, (1998)

Kumar et al., “Antisense RNA: function and fate of duplex RNA in cells of higher eukaryotes”, Microbiol Mol Biol Rev. 62, p. 1415-1434, (1998)

Li et al., “RAC3, a steroid/nuclear receptor-associated coactivator that is related to SRC-1 and TIF2”, Proc Natl Acad Sci USA, 94:8479-84 (1997)

Ma et al., “Multiple signal input and output domains of the 160-kilodalton nuclear receptor coactivator proteins”, Mol Cell Biol, 19:6164-73 (1999)

McEwan et al., “Interaction of the human androgen receptor transactivation function with the general transcription factor TFIIF”, Proc Natl Acad Sci USA, 94:8485-90 (1997)

Meiri et al., “Memory and long-term potentiation (LTP) dissociated: normal spatial memory despite CAl LTP elimination with Kv1.4 antisense” PNAS 95, p. 15037-15042, (1998)

Nolan G P, Ghosh S, Liou H C, Tempst P and Baltimore D., “DNA binding and I kappa B inhibition of the cloned p65 subunit of NF_kappa B, a rel-related polypeptide”, Cell 64 (5), 961-969 (1991)

Onate et al., “Sequence and characterization of a coactivator for the steroid hormone receptor superfamily”, Science, 270:1354-7 (1995)

Ogawa, S., Inoue, S., Watanabe, T., Hiroi, H., Orimo, A., Hosoi, T., Ouchi, Y. and Muramatsu, M., “The complete primary structure of human estrogen receptor beta (hER beta) and its heterodimerization with ER alpha in vivo and in vitro”, Biochem. Biophys. Res. Commun. 243 (1), 122-126 (1998)

Pfahl et al., “Nucleotide sequence of cDNA encoding a novel human thyroid hormone receptor”, Nucleic Acids Res. 15 (22), 9613 (1987)

Picard et al., “Reduced levels of hsp90 compromise steroid receptor action in vivo”, Nature, 348:166-8 (1990)

Scheller et al., “Multiple receptor domains interact to permit, or restrict, androgen-specific gene activation”, J Biol Chem, 273:24216-22 (1998)

Schroer et al., “Cloning and characterization of UXT, a novel gene in human Xp11, which is widely and abundantly expressed in tumor tissue”, Genomics, 56:340-3 (1999)

Segnitz et al., “The function of steroid hormone receptors is inhibited by the hsp90-specific compound geldanamycin”, J Biol Chem, 272:18694-701 (1997)

Shoji et al., “Enhancement of anti-herpetic activity of antisense phosphorothioate oligonucleotides 5′ end modified with geraniol” J. Drug Target 5, p. 261-73, (1998)

Smith et al., “CREB binding protein acts synergistically with steroid receptor coactivator-1 to enhance steroid receptor-dependent transcription”, Proc Natl Acad Sci USA, 93:8884-8 (1996)

Soukchareun et al., “Use of Nalpha-Fmoc-cysteine(S-thiobutyl) derivatized oligodeoxynucleotides for the preparation of oligodeoxynucleotide-peptide hybrid molecules”, Bioconjug. Chem. 9, p. 466-75, (1998)

Stix, “Shutting down a gene. Antisense drug wins approval”, Sci. Amer. 279, p. 46, 50, (1998)

Szapary et al., “Opposing effects of corepressor and coactivators in determining the dose-response curve of agonists, and residual agonist activity of antagonists, for glucocorticoid receptor-regulated gene expression” Mol Endocrinol, 13:2108-21 (1999)

Torchia et al., “The transcriptional co-activator p/CIP binds CBP and mediates nuclear-receptor function”, Nature, 387:677-84 (1997)

Trapman et al., “The androgen receptor in prostate cancer”, Pathol Res Pract 192:752-60 (1996)

Visakorpi et al., “Genetic changes in primary and recurrent prostate cancer by comparative genomic hybridization” Cancer Res, 55:342-7 (1995a)

Visakorpi et al., “In vivo amplification of the androgen receptor gene and progression of human prostate cancer”, Nat Genet, 9:401-6 (1995b)

Voegel et al., “TIF2, a 160 kDa transcriptional mediator for the ligand-dependent activation function AF-2 of nuclear receptors”, Embo J, 15:3667-75 (1996)

Wang, J. Controlled Release 53, p. 39-48, (1998)

Wilson et al., “Molecular analysis of the androgen receptor”, Ann NY Acad Sci, 637:56-63 (1991)

Yasuda et al., “ATBF1, a multiple-homeodomain zinc finger protein, selectively down-regulates AT-rich elements of the human alpha-fetoprotein gene”, Mol Cell Biol, 14:1395-401 (1994)

Yeh et al., Inhibition of BMP receptor synthesis by antisense oligonucleotides attenuates OP-1 action in primary cultures of fetal rat calvaria cells”, J. Done Miner. Res. 13:1870-1879, (1998)

Yeh et al., “Cloning and characterization of a specific coactivator, ARA70, for the androgen receptor in human prostate cells”, Proc Natl Acad Sci USA, 93:5517-21 (1996)

What is claimed is:

1. A method for treating an androgen dependent disease, comprising administering to a patent in need thereof an effective amount of a molecule which comprises the antigen-binding portion of an antibody specific for a polypeptide consisting of the amino acid sequence of SEQ ID NO:4.

2. The method of claim 1, wherein the molecule is a monoclonal antibody.

3. The method of claim 1, wherein the androgen dependent disease is prostate cancer.

4. The method of claim 1, wherein the androgen dependent disease is benign prostatic hyperplasia.

5. The method of claim 1, wherein the androgen dependent disease is androgen dependent hair loss.


SEQUENCE LISTING (20 sequences)

SEQ ID NO: 1 - DNA, 474 bp, human
atggcgacgccccctaagcggcgggcggtggaggccacgggggagaaagtgctgcgctacgagaccttcatcagtgacgtgctgcagcgggacttgcgaaaggtgctggaccatcgagacaaggtatatgagcagctggccaaataccttcaactgagaaatgtcattgagcgactccaggaagctaagcactcggagttatatatgcaggtggatttgggctgtaacttcttcgttgacacagtggtcccagatacttcacgcatctatgtggccctgggatatggttttttcctggagttgacactggcagaagctctcaagttcattgatcgtaagagctctctcctcacagagctcagcaacagcctcaccaaggactccatgaatatcaaagcccatatccacatgttgctagaggggcttagagaactacaaggcctgcagaatttcccagagaagcctcaccattga

SEQ ID NO: 2 - protein, 157 aa, human
MATPPKRRAVEATGEKVLRYETFISDVLQRDLRKVLDHRDKVYEQLAKYLQLRNVIERLQEAKHSELYMQVDLGCNFFVDTVVPDTSRIYVALGYGFFLELTLAEALKFIDRKSSLLTELSNSLTKDSMNIKAHIHMLLEGLRELQGLQNFPEKPHH

SEQ ID NO: 3 - DNA, 1097 bp, human
aaatgcacaacccggacggaagtgcctctccgacagcagatccaggctcggagctccagacgctgggacaggccgcccgcagaccacccccgccgcgcgcgggacacgacgccccccgcaggacacgcccatcagcccggaaacccctgagctgcttctcccggaggccgatgcccacccgggagcccccaaagactcgcggctcccgggggcacctgcatactcacccgcctgggcctgggcccccgctgcagggactggcgccccgaggcctcaaaaccagcgccccccgccctccgtgccagccccagccgggaccccacaaggcaaagaccaagaagattgtgtttgaggatgagttgctctcccaggccctcctgggcgccaagaagcctattggagccatccctaaggggcataagcctaggccccacccagtgcccgactatgagcttaagtacccgccagtgagcagtgagagggaacggagccgctatgtcgcagtgttccaggaccagtacggagagttcttggagctccagcacgaggtggggtgtgcacaggcaaagctcaggcagctggaggccctgctgagctccctgcccccaccccaaagccagaaggaggcccaagttgcagcccgggtttggagggagtttgagatgaagcgaatggatcctggcttcctggacaagcaggctcgctgccactacctgaagggtaaactgaggcatctcaagactcagatccagaaattcgatgaccaaggagacagcgagggctccgtgtacttctaagtgcccctgcagatgggcagagggatgcatggggatgcaggtcccttgcatttcttggtatctctcagcttttcctcttgcagctccccctaccaggggtcgctttctcctggattgcaaatgcctcttcagtttggactcagctctgacagcccctcctccaggaaggccttccaggacttcctcctctgggtcctctagctctgaccctacagggactccagatctcaacctgttccctggaagtagggcctgctctccatcccagtgaaataaacatgtattagacacctaaaaaaa

SEQ ID NO: 4 - protein, 264 aa, Human
MHNPDGSASPTADPGSELQTLGQAARRPPPPRAGHDAPRRTRPSARKPLSCFSRRPMPTREPPKTRGSRGHLHTHPPGPGPPLQGLAPRGLKTSAPRPPCQPQPGPHKAKTKKIVFEDELLSQALLGAKKPIGAIPKGHKPRPHPVPDYELKYPPVSSERERSRYVAVFQDQYGEFLELQHEVGCAQAKLRQLEALLSSLPPPQSQKEAQVAARVWREFEMKRMDPGFLDKQARCHYLKGKLRHLKTQIQKFDDQGDSEGSVYF

SEQ ID NO: 5 - DNA, 517 bp, Human
  misc_feature (65)..(65): n at position is unknown.
gaacggcacgagggcgcgccacgcgcgggaagcggcgcgcggagcgcgcgcggcgggccgcgcanccgagggagccgagcgcccgmacgcgcccgagcggacasacgccagagccgcgccccgggccgagcgcagcgcgccggccgssygggccgccaggggcgcgcgcggcggagcgcggggcgcgmgaaaaggggcccggcggagaccaagggcaggcgcggcccgcaagggcgccggggaaggcgcccggcaaggaggcggacaagcggagcaggccaacgagacgcgcgcacccacacacgagcgcgagccgccacaacaccacacccggcccaaggagaacagcacgccaacgcgccagycacggcgggcacgggaggcgggccacacacagcggccccgccaaggcacggcgcacggcacaagggcaccacgccagacaagcgaggaggcagcacgccgagaccggccggagggccgcgaccgccggagaaaaggaacagagagcccccca

SEQ ID NO: 6 - protein, 189 aa, Human
EFGTRARFTRGKSALLERALARPRTEVSLSAFALLSPSWYSTARAVFSVAELQSRLAALGRQVGARVLDALVAREKGARRETKVLGALLFVKGAVWKALFGKEADKLEQANDDARTFYIIEREPLINTYISVPKENSTLNCASFTAGIVEAVLTHSGFPAKVTAHWHKGTTLMIKFEEAVIARDRLEGR

SEQ ID NO: 7 - DNA, 126 bp, Human
gaattcggcacgaggctcaagccctacgtgagctacctcgcccctgagagcgaggagacgcccctgacggccgcgcagctcttcagcaagccgttggcgccttgccatcgaaaaggacttcaagga

SEQ ID NO: 8 - protein, 42 aa, Human
EFGTRLKPYVSYLAPESEETPLTAAQLFSKPLAPCHRKGLQG

SEQ ID NO: 9 - DNA, 678 bp, Human
  misc_feature (651)..(651): n at position is unknown.
gaattcggcacgaggattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcgcagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttcctctacacttatcatcttcacaattctaattctactgactatcctagaaatcgctgtcgccttaatccaagcctacgttttcacacttctagtaagcctntactgnacgacaacacataaaaaaaaa

SEQ ID NO: 10 - protein, 60 aa, Human
EFGTRIHCPHNPRPTRRSTDHSISPSIDPHLQISHQQPTNHHPTMTNQTNLKTNDNHTQH

SEQ ID NO: 11 - DNA, 1918 bp, Human
gaattccaatgtggtaaagtcttcgctcaaacatcacaacttgcaaggcattggagagttcatactggagaaaaaccttacaagtgtaatgactgtggcagagcctttagtgatcgttcaagcctaacttttcatcaggcaatacatactggagagaaaccttacaaatgtcatgaatgcggcaaggtttttaggcacaattcataccttgcaactcatcggcgaattcatactggagagaaaccttacaagtgtaatgagtgtgggaaagcctttagtatgcattcaaacctaactacccataaggtcatccatactggagagaagccttacaaatgtaatcaatgtggcaaggtcttcactcagaactcacaccttgcaaatcatcaaaggactcacaccggagagaaaccttaccgatgcaatgagtgtgggaaagccttcagtgttcgttcaagcctaaccacccatcaggcaatccatactgggaaaaaaccttacaaatgtaatgaatgtggcaaggtctttactcaaaatgctcacctggcaaatcaccgaagaattcatactggggagaaaccttacaggtgtacagagtgtgggaaagcctttagggtaagatcaagtctaactacccatatggcaatccacactggagaaaagcgttacaaatgtaatgagtgtggcaaggtcttcaggcagagttcaaatcttgcaagtcatcacagaatgcataccggagagaaaccttacaaatgagtgtggtgaggtcattaggtacaattcactcctttcacatcagttaatttcattcttgacagaatccttacaaatgtagtgacagtggccaatccctcatgagttgaagcattaatagatatgagaggccataagcaagagacatcatgtaaacatatgtggcagagggtctatccaggcctcgcaggttactaggcatcaagatttatatctttgatgaaacgaaacaaatgtaatatgcatcctgaggccattacccagtgaccgatggtaagtgaggattcctaggaggaataacagtctctggtttccctgtttgcctttgatattatacactgtagaatactcacaagtccaaatatgctaaaaattatatatttttaactcacatacgaaaaggttgcaggatatttgtaggcagtcagttaccttcaccttatgaaatgtttcactgagttatttgaggttttttggaaagcctactattgcgtttcaatgtgaactttgaaatcttattgtgcatccttacacaccttccatggtgctttcttggaaagatcattgggatggaaggatcattgattgggtgaagatcattgattaggtgaaggattatttctatccaatttgtgaagaaggaggactttgcttttaaaattaagtatcatctgaattagcatttgggagtggcgaaaaacaatgtaaaactatgatgtcactcaccattctgataatgttcagggtgcctttctcctaccaggagagtactgtggcttagaggaaagaaatggtctatcaactgaacatgaaatggagcaggccaagaccttaggacattgggatttttgtgggaggagagtaataggtaattagacactgattgtgtggtagaaatactgcaggggaaaaggtcgccctcttatgcatcaaagagcaatacctgttgtttagcaaagagtgatgaaaaattgatcttgttttgaaattgaagagagaggccaggcgcggtggctcacacctgtaatcccagcactttgggaggctgaggcaggtggatcacctgaggtcgggagttcgagaccagcctgaccaacatggagaaaccccaattgtactaaaaatacaaaattagccgggcgtggtggcaggtgcggaattc

SEQ ID NO: 12 - protein, 252 aa, Human
EFQCGKVFAQTSQLARHWRVHTGEKPYKCNDCGRAFSDRSSLTFHQAIHTGEKPYKCHECGKVFRHNSYLATHRRIHTGEKPYKCNECGKAFSMHSNLTTHKVIHTGEKPYKCNQCGKVFTQNSHLANHQRTHTGEKPYRCNECGKAFSVRSSLTTHQAIHTGKKPYKCNECGKVFTQNAHLANHRRIHTGEKPYRCTECGKAFRVRSSLTTHMAIHTGEKRYKCNECGKVFRQSSNLASHHRMHTGEKPYK

SEQ ID NO: 13 - DNA, 8588 bp, Human
cgcggcccgagcgcctcttttcgggattaaaagcgccgccagctcccgccgccgccgccgtcgccagcagcgccgctgcagccgccgccgccggagaagcaaccgctgggcggtgagatccccctagacatgcggctcgggggcgggcagctggtgtcagaggagctgatgaacctgggcgagagcttcatccagaccaacgacccgtcgctgaagctcttccagtgcgccgtctgcaacaagttcacgacggacaacctggacatgctgggcctgcacatgaacgtggagcgcagcctgtcggaggacgagtggaaggcggtgatgggggactcataccagtgcaagctctgccgctacaacacccagctcaaggccaacttccagctgcactgcaagacagacaagcacgtgcagaagtaccagctggtggcccacatcaaggagggcggcaaggccaacgagtggaggctcaagtgtgtggccatcggcaaccccgtgcacctcaagtgcaacgcctgtgactactacaccaacagcctggagaagctgcggctgcacacggtcaactccaggcacgaggccagcctgaagttgtacaagcacctgcagcagcatgagagtggtgtagaaggtgagagctgctactaccactgcgttctgtgcaactactccaccaaggccaagctcaacctcatccagcatgtgcgctccatgaagcaccagcgaagcgagagcctgcgaaagctgcagcggctgcagaagggccttccagaggaggacgaggacctggggcagatcttcaccatccgcaggtgcccctccacggacccagaagaagccattgaagatgttgaaggacccagtgaaacagctgctgatccagaggagcttgctaaggaccaagagggcggagcatcgtccagccaagcagagaaggagctgacagattctcctgcaacctccaaacgcatctccttcccaggtagctcagagtctcccctctcttcgaagcgaccaaaaacagctgaggagatcaaaccggagcagatgtaccagtgtccctactgcaagtacagtaatgccgatgtcaaccggctccgggtgcatgccatgacgcagcactcggtgcaacccatgcttcgctgccccctgtgccaggacatgctcaacaacaagatccacctccagctgcacctcacccacctccacagcgtggcacctgactgcgtggagaagctcattatgacggtgaccacccctgagatggtgatgccaagcagcatgttcctcccagcagctgttccagatcgagatgggaattccaatttggaagaggcaggaaagcagcctgaaacctcagaggatctgggaaagaacatcttgccatccgcaagcacagagcaaagcggagatttgaaaccatcccctgctgacccaggctctgtgagagaagactcaggcttcatctgctggaagaaggggtgcaaccaggttttcaaaacttctgctgcccttcagacgcattttaatgaagtgcatgccaagaggcctcagctgccggtgtcagatcgccatgtgtacaagtaccgctgtaatcagtgtagcctggccttcaagaccattgaaaagttgcagctccattctcagtaccatgtgatcagagctgccaccatgtgctgtctttgtcagcgcagtttccgaactttccaggctctgaagaagcaccttgagacaagccacctggagctgagtgaggctgacatccaacagctttatggtggcctgctggccaatggggacctcctggcaatgggagaccccactctggctgaggaccataccataattgttgaggaagacaaggaggaagagagtgacttggaagataaacagagcccaacgggcagtgactctgggtcagtacaagaagactcgggctcagagccaaagagagctctgcctttcagaaaaggtcccaattttactatggaaaagttcctagacccttctcgcccttacaagtgtaccgtctgcaaggaatctttcactcaaaagaatatcctgctagtacactacaattctgtctcccacctgcataagttaaagagagcccttcaagaatcagcaaccggtcagccagaacccaccagcagcccagacaacaaaccttttaagtgtaacacttgtaatgtggcctacagccagagttccactctggagatccatatgaggtctgtgttacatcaaaccaaggcccgggcagccaagctggaggctgcaagtggcagcagcaatgggactgggaacagcagcagtatttccttgagctcctccacgccaagtcctgtgagcaccagtggcagtaacacctttaccacctccaatccaagcagtgctggcattgctccaagctctaacttactaagccaagtgcccactgagagtgtagggatgccacccctggggaatcctattggtgccaacattgcttccccttcagagcccaaagaggccaatcggaagaaactggcagatatgattgcatccaggcagcagcaacaacagcagcagcaacagcaacaacaacaacaacaacaacaacaacaagcacaaacgctggcccaggcccaggctcaagttcaagctcacctgcagcaggagctgcagcaacaggctgccctgatccagtctcagctgtttaaccccaccctccttcctcacttccccatgacaactgagaccctgctgcaactacagcagcagcagcacctcctcttccctttctacatccccagtgctgagttccagcttaaccccgaggtgagcttgccagtgaccagtggggcactgacactgactgggacaggcccaggcctgctggaagatctgaaggctcaggttcaggtcccacagcagagccatcagcagatcttgccgcagcagcagcagaaccaactctctatagcccagagtcactctgccctccttcagccaagccagcaccccgaaaagaagaacaaattggtcatcaaagaaaaggaaaaagaaagccagagagagagggacagcgccgaggggggagagggcaacaccggtccgaaggaaacactgccagatgccttgaaggccaaagagaagaaagagttggcaccagggggtggttctgagccttccatgctccctccacgcattgcttcagatgccagagggaacgccaccaaggccctgctggagaactttggctttgagttggtcatccagtataatgagaacaagcagaaggtgcagaaaaagaatgggaagactgaccagggagagaacctggaaaagctcgagtgtgactcctgcggcaagttgttttccaacatcttgattttaaagagtcatcaagagcacgttcatcagaattactttcctttcaaacagctcgagaggtttgccaaacagtacagagaccactacgataaactgtacccactgaggccccagaccccagagccaccaccacctccccctccaccccctccacccccacttccggcagcgccgcctcagccggcgtccacaccagccatccccgcatcagccccacccatcacctcacctacaattgcaccggcccagccatcagtgccgctcacccagctctccatgccgatggagctgcccatcttctcgccgctgatgatgcagacgatgccgctgcagaccttgccggctcagctacccccgcagctgggacctgtggagcctctgcctgcggacctggcccaactctaccagcatcagctcaatccaaccctgctccagcagcagaacaagaggcctcgcaccaggatcacagatgatcagctccgagtcttgcggcaatattttgacattaacaactcccccagtgaagagcaaataaaagagatggcagacaagtccgggttgccccagaaagtgatcaagcactggttcaggaacactctcttcaaagagaggcagcgtaacaaggactccccttacaacttcagtaatcctcctatcaccagcctggaggagctcaagattgactcccggcccccttcgccggaacctccaaagcaggagtactggggaagcaagaggtcttcaagaacaaggtttacggactaccagctgagggtcttacaggacttcttcgatgccaatgcttacccaaaggatgatgaatttgagcaactctctaatttactgaaccttccaacccgagtgatagtggtgtggtttcagaatgcccgacagaaggccaggaagaattatgagaatcagggagagggcaaagatggagagcggcgtgagcttacaaatgatagatacattcgaacaagcaacttgaactaccagtgcaaaaaatgtagcctggtgtttcagcgcatctttgatctcatcaagcaccagaagaagctgtgttacaaggatgaggatgaggaggggcaggacgacagccaaaatgaggattccatggatgccatggaaatcctgacgcctaccagctcatcctgcagtaccccgatgccctcacaggcttacagcgccccagcaccatcagccaataatacagcttcctccgctttcttgcagcttacagcggaggctgaggaactggccaccttcaattcaaaaacagaggcaggcgatgagaaaccaaagctggcggaagctcccagtgcacagccaaaccaaacccaagaaaagcaaggacaaccaaagccagagctgcagcagcaagagcagcccgagcagaagaccaacactccccagcagaagctcccccagctggtgtccctgccttcgttgccacagcctcctccacaagcgccccctccacagtgccccttaccccagtcgagccccagtccttcccagctctcccacctgcccctcaagcccctccacacatcaactcctcaacagctcgcaaacctacctcctcagctaatcccctaccagtgtgaccagtgtaagttggcatttccgtcatttgagcactggcaggagcatcagcagctccacttcctgagcgcgcagaaccagttcatccacccccagtttttggacaggtccctggatatgcctttcatgctctttgatcccagtaacccactcctggccagccagctgctctctggggccatacctcagattccagcaagctcagccacttctccttcaactccaacctccacaatgaacactctcaagaggaagctggaggaaaaggccagtgcaagccctggcgaaaacgacagtgggacaggaggagaagagcctcagagagacaagcgtttgagaacaaccatcacaccggaacaactagaaattctctaccagaagtatctactggattccaatccgactcgaaagatgttggatcacattgcacacgaggtgggcttgaagaaacgtgtggtacaagtctggtttcagaacacccgagctcgggaaaggaaaggacagttccgggctgtaggcccagcgcaggcccacaggagatgccctttttgcagagcgctcttcaaagccaagactgctcttgaggctcatatccggtcccgtcactggcatgaagccaagagagctggctacaacctaactctgtctgcgatgctcttagactgtgatgggggactccagatgaaaggagatatttttgacggaactagcttttcccacctacccccaagcagtagtgatggtcagggtgtccccctctcacctgtgagtaaaaccatggaattgtcacccagaactcttctaagcccttcctccattaaggtggaagggattgaagactttgaaagcccctccatgtcctcagttaatctaaactttgaccaaactaagctggacaacgatgactgttcctctgtcaacacagcaatcacagataccacaactggagacgagggcaacgcagataacgacagtgcaacgggaatagcaactgaaaccaaatcctcttctgcacccaacgaagggttgaccaaagcggccatgatggcaatgtctgagtatgaagatcggttgtcatctggtctggtcagcccggccccgagcttttatagcaaggaatatgacaatgaaggtacagtggactacagtgaaacctcaagccttgcagatccctgctccccgagtcctggtgcgagtggatctgcaggcaaatctggtgacagcggggatcggcctgggcagaaacgttttcgcactcaaatgaccaatctgcagctgaaggtcctcaagtcatgctttaatgactacaggacacccactatgctagaatgtgaggtcctgggcaatgacattggactgccaaagagagtcgttcaggtctggttccagaatgcccgggcaaaagaaaagaagtccaagttaagcatggccaagcattttggtataaaccaaacgagttatgagggacccaaaacagagtgcactttgtgtggcatcaagtacagcgctcggctgtctgtacgtgaccatatcttttcccaacagcatatctccaaagttaaagacaccattggaagccagctggacaaggagaaagaatactttgacccagccaccgtacgtcagttgatggctcaacaagagttggaccggattaaaaaggccaacgaggtccttggactggcagctcagcagcaagggatgtttgacaacacccctcttcaggcccttaaccttcctacagcatatccagcgctccagggcattcctcctgtgttgctcccgggcctcaacagcccctccttgccaggctttactccatccaacacagctttaacgtctcctaagccgaacttgatgggtctgcccagcacaactgttccttcccctggcctccccacttctggattaccaaataaaccgtcctcagcgtcgctgagctccccaaccccagcacaagccacgatggcgatgggccctcagcaacccccccagcagcagcagcagcagcagcaaccacaggtgcagcagcctcccccgccgccagcagcccagccgccacccacaccacagctcccactgcaacagcagcagcaacgcaaggacaaagacagtgagaaagtaaaggagaaggaaaaggcacacaaagggaaaggggaacccctgcctgtccccaagaaggagaaaggagaggcccccacggcaactgcagccacgatctcagccccgctgcccaccatggagtatgcggtagaccctgcacagctgcaggccctgcaggccgcgttgacttcggaccccacagcattgctcacaagccagttccttccttactttgtaccaggcttttctccttattatgctccccagatccctggcgccctgcagagcgggtacctgcagcctatgtatggcatggaaggcctgttcccctacagccctgcactgtcgcaggccctgatggggctgtccccaggctccctactgcagcagtaccagcaataccagcagagtctgcaggaggcaattcagcagcagcagcagcaaaaagtgcagcagcagcagcccaaagcaagccaaaccccagtcccccccggggctccttccccagacaaagaccctgccaaagaatcccccaaaccagaagaacagaaaaacaccccccgtgaggtgtcccccctcctgccgaaactccctgaagagccagaagcagaaagcaaaagtgcggactccctctacgaccccttcattgttccaaaggtgcagtacaagttggtctgccgcaagtgccaggcgggcttcagcgacgaggaggcagcgaggagccacctgaagtccctctgcttcttcggccagtctgtggtgaacctgcaagagatggtgcttcacgtccccaccggcggcggcggcggtggcagtggcggcggcggcggcggtggcggcggcggcggcggcggcggcggcggctcgtaccactgcctggcgtgcgagagcgcgctctgtggggaggaagctctgagtcaacatctcgagtcggccttgcacaaacacagaacaatcacgagagcagcaagaaacgccaaagagcaccctagtttattacctcactctgcctgcttccccgatcctagcaccgcatctacctcgcagtctgccgctcactcaaacgacagcccccctcccccgtcggccgccgccccctcctccgcttccccccacgcctccaggaagtcttggccgcaagtggtctcccgggcttcggcagcgaagcccccttcttttcctcctctctcctcatcttcaacggttacctcaagttcatgcagcacctcaggggttcagccctcgatgccaacagacgactattcggaggagtctgacacggatctcagccaaaagtccgacggaccggcgagcccggtggagggtcccaaagaccccagctgccccaaggacagtggtctgaccagtgtaggaacggacaccttcagattgtaagctttgaagatgaacaatacaaacaaatgaatttaaatacaaaaattaataacaaaccaatttcaaaaatagactaactgcaattccaaagcttctaaccaaaaaac

SEQ ID NO: 14 - protein, 2783 aa, Human
MRLGGGQLVSEELMNLGESFIQTNDPSLKLFQCAVCNKFTTDNLDMLGLHMNVERSLSEDEWKAVMGDSYQCKLCRYNTQLKANFQLHCKTDKHVQKYQLVAHIKEGGKANEWRLKCVAIGNPVHLKCNACDYYTNSLEKLRLHTVNSRHEASLKLYKHLQQHESGVEGESCYYHCVLCNYSTKAKLNLIQHVRSMKHQRSESLRKLQRLQKGLPEEDEDLGQIFTIRRCPSTDPEEAIEDVEGPSETAADPEELAKDQEGGASSSQAEKELTDSPATSKRISFPGSSESPLSSKRPKTAEEIKPEQMYQCPYCKYSNADVNRLRVHAMTQHSVQPMLRCPLCQDMLNNKIHLQLHLTHLHSVAPDCVEKLIMTVTTPEMVMPSSMFLPAAVPDRDGNSNLEEAGKQPETSEDLGKNILPSASTEQSGDLKPSPADPGSVREDSGFICWKKGCNQVFKTSAALQTHFNEVHAKRPQLPVSDRHVYKYRCNQCSLAFKTIEKLQLHSQYHVIRAATMCCLCQRSFRTFQALKKHLETSHLELSEADIQQLYGGLLANGDLLAMGDPTLAEDHTIIVEEDKEEESDLEDKQSPTGSDSGSVQEDSGSEPKRALPFRKGPNFTMEKFLDPSRPYKCTVCKESFTQKNILLVHYNSVSHLHKLKRALQESATGQPEPTSSPDNKPFKCNTCNVAYSQSSTLEIHMRSVLHQTKARAAKLEAASGSSNGTGNSSSISLSSSTPSPVSTSGSNTFTTSNPSSAGIAPSSNLLSQVPTESVGMPPLGNPIGANIASPSEPKEANRKKLADMIASRQQQQQQQQQQQQQQQQQQQAQTLAQAQAQVQAHLQQELQQQAALIQSQLFNPTLLPHFPMTTETLLQLQQQQHLLFPFYIPSAEFQLNPEVSLPVTSGALTLTGTGPGLLEDLKAQVQVPQQSHQQILPQQQQNQLSIAQSHSALLQPSQHPEKKNKLVIKEKEKESQRERDSAEGGEGNTGPKETLPDALKAKEKKELAPGGGSEPSMLPPRIASDARGNATKALLENFGFELVIQYNENKQKVQKKNGKTDQGENLEKLECDSCGKLFSNILILKSHQEHVHQNYFPFKQLERFAKQYRDHYDKLYPLRPQTPEPPPPPPPPPPPPLPAAPPQPASTPAIPASAPPITSPTIAPAQPSVPLTQLSMPMELPIFSPLMMQTMPLQTLPAQLPPQLGPVEPLPADLAQLYQHQLNPTLLQQQNKRPRTRITDDQLRVLRQYFDINNSPSEEQIKEMADKSGLPQKVIKHWFRNTLFKERQRNKDSPYNFSNPPITSLEELKIDSRPPSPEPPKQEYWGSKRSSRTRFTDYQLRVLQDFFDANAYPKDDEFEQLSNLLNLPTRVIVVWFQNARQKARKNYENQGEGKDGERRELTNDRYIRTSNLNYQCKKCSLVFQRIFDLIKHQKKLCYKDEDEEGQDDSQNEDSMDAMEILTPTSSSCSTPMPSQAYSAPAPSANNTASSAFLQLTAEAEELATFNSKTEAGDEKPKLAEAPSAQPNQTQEKQGQPKPELQQQEQPEQKTNTPQQKLPQLVSLPSLPQPPPQAPPPQCPLPQSSPSPSQLSHLPLKPLHTSTPQQLANLPPQLIPYQCDQCKLAFPSFEHWQEHQQLHFLSAQNQFIHPQFLDRSLDMPFMLFDPSNPLLASQLLSGAIPQIPASSATSPSTPTSTMNTLKRKLEEKASASPGENDSGTGGEEPQRDKRLRTTITPEQLEILYQKYLLDSNPTRKMLDHIAHEVGLKKRVVQVWFQNTRARERKGQFRAVGPAQAHRRCPFCRALFKAKTALEAHIRSRHWHEAKRAGYNLTLSAMLLDCDGGLQMKGDIFDGTSFSHLPPSSSDGQGVPLSPVSKTMELSPRTLLSPSSIKVEGIEDFESPSMSSVNLNFDQTKLDNDDCSSVNTAITDTTTGDEGNADNDSATGIATETKSSSAPNEGLTKAAMMAMSEYEDRLSSGLVSPAPSFYSKEYDNEGTVDYSETSSLADPCSPSPGASGSAGKSGDSGDRPGQKRFRTQMTNLQLKVLKSCFNDYRTPTMLECEVLGNDIGLPKRVVQVWFQNARAKEKKSKLSMAKHFGINQTSYEGPKTECTLCGIKYSARLSVRDHIFSQQHISKVKDTIGSQLDKEKEYFDPATVRQLMAQQELDRIKKANEVLGLAAQQQGMFDNTPLQALNLPTAYPALQGIPPVLLPGLNSPSLPGFTPSNTALTSPKPNLMGLPSTTVPSPGLPTSGLPNKPSSASLSSPTPAQATMAMGPQQPPQQQQQQQQPQVQQPPPPPAAQPPPTPQLPLQQQQQRKDKDSEKVKEKEKAHKGKGEPLPVPKKEKGEAPTATAATISAPLPTMEYAVDPAQLQALQAALTSDPTALLTSQFLPYFVPGFSPYYAPQIPGALQSGYLQPMYGMEGLFPYSPALSQALMGLSPGSLLQQYQQYQQSLQEAIQQQQQQKVQQQQPKASQTPVPPGAPSPDKDPAKESPKPEEQKNTPREVSPLLPKLPEEPEAESKSADSLYDPFIVPKVQYKLVCRKCQAGFSDEEAARSHLKSLCFFGQSVVNLQEMVLHVPTGGGGGGSGGGGGGGGGGGGGGGGSYHCLACESALCGEEALSQHLESALHKHRTITRAARNAKEHPSLLPHSACFPDPSTASTSQSAAHSNDSPPPPSAAAPSSASPHASRKSWPQVVSRASAAKPPSFPPLSSSSTVTSSSCSTSGVQPSMPTDDYSEESDTDLSQKSDGPASPVEGPKDPSCPKDSGLTSVGTDTFRL

SEQ ID NO: 15 - DNA, 30 bp, Artificial Sequence
  synthetic
agatcttaagcagaaatgattgcaccattg

SEQ ID NO: 16 - DNA, 28 bp, Artificial Sequence
  synthetic
gtagataaaggtgtgtgtcactgagctc

SEQ ID NO: 17 - DNA, 19 bp, Artificial Sequence
  synthetic
ttggggttattcgcaacgg

SEQ ID NO: 18 - DNA, 35 bp, Artificial Sequence
  synthetic
gaactggatccctgctcatataccttgtctcgatg

SEQ ID NO: 19 - DNA, 26 bp, Artificial Sequence
  synthetic
gaactggatccaccaaggactccatg

SEQ ID NO: 20 - DNA, 18 bp, Artificial Sequence
  synthetic
cggaattagcttggctgc